(19) United States
(12) Patent Application Publication
(10) Pub. No.: US 2004/0006043 A1
(43) Pub. Date: Jan. 8, 2004

(54) Methods, pharmaceutical compositions and pharmaceutical kits for enhancing the therapeutic efficiency of cancer chemotherapeutic agents

(75) Inventors: Rimona Margalit, Givataim (IL); Dan Peer, Kiryat Ono (IL)

Correspondence Address: G.E. EHRLICH (1995) LTD., c/o ANTHONY CASTORINA, SUITE 207, 2001 JEFFERSON DAVIS HIGHWAY, ARLINGTON, VA, 22202, US

(73) Assignee: RAMOT UNIVERSITY AUTHORITY FOR APPLIED RESEARCH & INDUSTRIAL DEVELOPMENT LTD.

(21) Appl. No.: 10/187,362

(22) Filed: Jul. 2, 2002

(51) Int. Cl. A61K 31/728; A61K 38/12; A61K 31/405; A61K 31/455; A61K 31/4706; A61K 31/554; A61K 31/56; A61K 9/127

(52) U.S. Cl. 514/54; 424/450; 514/211.07; 514/9; 514/312; 514/171; 514/414; 514/355

(57) ABSTRACT

A pharmaceutical composition which comprises a chemotherapeutically effective amount of one or more chemotherapeutic agent(s), being entrapped in a drug carrier, and a chemosensitizing effective amount of one or more chemosensitizing agent(s) and/or a chemoprotecting effective amount of one or more chemoprotecting agent(s) and methods of using same in cancer therapy.

### FIELD AND BACKGROUND OF THE INVENTION

The present invention relates generally to the field of oncology, and provides methods, pharmaceutical compositions and pharmaceutical kits for enhancing the therapeutic efficiency of cancer chemotherapeutic agents. More particularly, the present invention relates to pharmaceutical compositions which comprise a chemotherapeutic agent entrapped in a site-adherent site-retained carrier that is targeted to tumor cells, a chemosensitizing agent and/or a chemoprotective agent. These compositions are useful in the treatment of drug-resistant tumors, especially in cases of Multidrug Resistance (MDR).

Many of the most prevalent forms of human cancer resist effective chemotherapeutic intervention. Some tumor populations, especially adrenal, colon, jejunal, kidney and liver carcinomas, appear to have drug-resistant cells at the outset of treatment (Barrows, L. R., “Antineoplastic and Immunoactive Drugs”, Chapter 75, pp 1236-1262, in: Remington: “The Science and Practice of Pharmacy, Mack Publishing Co. Easton, Pa., 1995). In other cases, a resistance-conferring genetic change occurs during treatment; the resistant daughter cells then proliferate in the environment of the drug. Whatever the cause, resistance often terminates the usefulness of an antineoplastic drug.

Clinical studies suggest that a common form of multidrug resistance in human cancers results from the expression of the MDR1 gene that encodes P-glycoprotein. This glycoprotein functions as a plasma membrane, energy-dependent, multidrug efflux pump that reduces the intracellular concentration of cytotoxic drugs. This mechanism of resistance may account for de novo resistance in common tumors, such as colon cancer and renal cancer, and for acquired resistance, as observed in common hematologic tumors such as acute nonlymphocytic leukemia and malignant lymphomas.

Although this type of drug resistance may be common, it is by no means the only mechanism by which cells become drug resistant. MDR is effected via an extrusion mechanism (Tan B., Piwnica-Worms D., Rater L., Multidug resistance transporters and modulation. Curr. Opin. Oncol, 2000 September; 12(5):450-8). The influx of chemotherapeutic drugs into cells is mainly by passive diffusion across the cell membrane, driven by the drug's electrochemical-potential gradient. In MDR cells there are energy-dependant extrusion channels that actively pump the drug out of the cells, reducing its intracellular concentration below lethal threshold. The first pump identified was named Pgp (for P-glycoprotein), the second was named MRP (for Multidrug Resistant associate Protein) and several more have been identified in recent years (Tan et al. 2000, ibid.). All of them are naturally occurring proteins, and their physiological roles are assumed to involve detoxification of cells. In MDR cells they are present, for reasons yet unknown, in a significantly higher number of copies than in other non-MDR cells. Hereinafter, these proteins acting as extrusion pumps in MDR cells are referred to, interchangeably, as “MDR pumps”, “MDR extrusion pumps” and “extrusion pumps”.

Hence, reduction of the multidrug resistance to chemotherapy is conventionally performed by inhibition of the extrusion pumps in order to halt the drug efflux and thus to increase the intracellular concentration of the cytotoxic drug. Such an inhibition can be achieved by a variety of mechanisms that span from a simple inhibition of the transporter to intervention at the level of the pump protein expression. These mechanisms typically involve chemical modification of the cancer treatment.

Chemical modification of cancer treatment involves the use of agents or maneuvers that are not cytotoxic in themselves, but modify the host or tumor so as to enhance anticancer therapy and/or selectively protect non-cancer cells from the effects of cytotoxic drugs. Such agents are called chemosensitizers and chemoprotectors, respectively.

Pilot studies using chemosensitizers indicate that these agents may reverse resistance in a subset of patients. These same preliminary studies also indicate that drug resistance is multifactorial, because not all drug-resistant patients have P-glycoprotein-positive tumor cells and only a few patients appear to benefit from the use of current chemosensitizers.

Chemosensitization research has centered on agents that reverse or modulate multidrug resistance in solid tumors, by modulating the activity of the MDR extrusion pumps. Chemosensitizers known to modulate the function of MDR extrusion pumps include: calcium channel blockers (verapamil, indicated for the treatment of hypertension), calmodulin inhibitors (trifluoperazine), indole alkaloids (reserpine), quinolines (quinine), lysosomotropic agents (chloroquine), steroids (progesterone), triparanol analogs (tamoxifen), detergents (cremophor EL), and cyclic Ode antibiotics (cyclospoes, indicated to prevent host vs. graft disease) (DeVita, V. T., et al., in Cancer, Principles & Practice of Oncology. 4th ed., J. B. Lippincott Co., Philadelphia, Pa., pp 2661-2664, 1993; Sonneveld P., Wiemer E. Inhibitors of multidrug resistance., Curr Opin Oncol November 1997; 9(6):543-8).

A review of studies in which chemosensitizing agents were used raise the following conclusions: (i) cardiovascular side effects associated with continuous, high-dose intravenous verapamil therapy are significant and dose-limiting; (ii) dose-limiting toxicities of the chemosensitizers, trifluoperazine and tamoxifen, was attributed to the inherent toxicity of the chemosensitizer and not due to enhanced chemotherapy toxicity; (iii) studies using high doses of cyclosporine A as a chemosensitizer found hyperbilirubinemia as a side effect; and (iv) further research is clearly needed to develop less toxic and more efficacious chemosensitizers to be used clinically (DeVita et al., 1993, ibid.).

For example, while verapamil is effective in hypertension treatment at the 24 μM range, for MDR reversal it requires the dose range of 10-15 μM, while at 6 μM it is already in the toxic domain.

Tumors that are considered drug-sensitive at diagnosis but acquire an MDR phenotype at relapse, pose an especially difficult clinical problem. At diagnosis, only a minority of tumor cells may express proteins that act as no extrusion pumps and treatment with chemotherapy provides a selection advantage for the few cells that are, for example, P-glycoprotein positive early in the course of disease. Another possibility is that natural-product-derived chemotherapy actually induces the expression of MDR1, leading to P-glycoprotein-positive tumors or other MDR pump-positive tumors at relapse. Using chemosensitizers early in the course of disease may prevent the emergence of MDR by eliminating the few cells that are MDR pump-positive at the beginning. In vitro studies have shown that selection of drug-resistant cells by combining verapamil and doxorubicin does prevent the emergence of MDR pump, but that an alternative drug resistance mechanism develops, which is secondary to altered topoisomerase II function (Dalton, W. S., Proc. Am. Assoc. Cancer Res. 31.:520, 1990).

Another prevalent problem that is associated with tumor chemotherapy is the appearance of adverse side affects upon the administration of a chemotherapeutic drug. These adverse side effects typically result from the fact that the chemotherapeutic agents, by attacking proliferating cancerous cells attack also normal, non-cancerous proliferating cells.

Hence, the chemical modification of cancer treatment often further involves the use of agents that reduce the cytotoxicity of cancer therapy toward normal cells. These agents are known as chemoprotective agents. Chemoprotective agents are typically characterized as agents that affect the proliferation and/or differentiation of normal cells and include, for example, bis-dioxopiperazine compounds, D-methionine, thiol- and selenol-containing compounds such as cysteine, cysteamine, glutathione, selenocysteine, selenocysteamine and WR compounds, amifostines, ergotamines, pyrridoxines, lymphotoxins, DPPE analogs, psychotropic agents and many more.

Some agents, such as lymphotoxins, are known to exert both chemosenstization and chemoprotection activity, when used in combination with a chemotherapeutic agent. Such agents are disclosed, for example, in WO 94/1 8961 and in U.S. Pat. No. 5,747,023, which are incorporated by reference as if fully set forth herein. Nevertheless, the therapeutic protocols disclosed in these references are not directed toward MDR chemotherapy.

Hence, all the presently known agents and methods for reducing drug resistance in cancer therapy have not provided yet a comprehensive and efficient solution to multidrug resistance, in clinic. As the presently known methods are generally directed toward inhibition of the drug efflux, by inhibiting the extrusion pumps, these methods are not directed toward increasing the drug influx and thereby shifting the influx/efflux balance of the drug under the dynamic conditions that prevail in vivo.

Addressing the drug influx impact on multidrug resistance in vivo is known to be a complicated issue. As the main mechanism by which the chemotherapeutic drug gains entry into the cell is by passive diffusion across the cell membrane, as is described hereinabove, the driving force for this diffusion is quite steady both in magnitude and in duration when studied in vitro with cell cultures. While during in vitro experiments the given drug dose and the time span of cell exposure to the drug is controlled by the research performer, the situation is quite different in vivo. In vivo, upon initiation of the administration of the drug, two opposing processes take place: accumulation of the drug at the extracellular region outside the tumor cell, due to drug administration, which eventually ceases upon termination of administration; and natural clearance processes that remove the drug from the vicinity of the tumor, during the course of drug administration and after its termination. A direct consequence of these opposing processes is that in vivo, the time span during which a driving force for drug influx exists is of short duration, and its magnitude has a peak value for an even shorter duration.

The impact of drug influx on the efficacy of chemotherapy have not been fully addressed so far, mainly due to the fact that most of the mechanistic studies on cancer therapy and drug resistance have been performed in vitro.

A more efficacious cancer therapy is therefore urgently needed to improve the outcome of chemotherapy, especially in cases of multidrug resistant tumors. Such therapy can be achieved by maintaining the intracellular levels of chemotherapeutic drugs above the lethal threshold.

The present invention addresses this issue and provides a new cancer therapy that is based on novel and unique combination of therapeutic agents, which simultaneously increase the drug influx and decrease the drug efflux, while exerting a synergistic effect.

### SUMMARY OF THE INVENTION

According to one aspect of the present invention there is provided a pharmaceutical composition which comprises a chemotherapeutically effective amount of one or more chemotherapeutic agents), being entrapped in a drug carrier, and a chemosensitizing effective amount of one or more chemosensitizing agent(s).

According to further features in preferred embodiments of the invention described below, the pharmaceutical composition further comprises a chemoprotective amount of one or more chemoprotecting agent(s).

According to another aspect of the present invention there is provided a pharmaceutical composition which comprises a chemotherapeutically effective amount of one or more chemotherapeutic agent(s), being entrapped in a drug carrier, and a chemoprotective effective amount of one or more chemoprotecting agent(s).

According to further features in preferred embodiments of the invention described below, the pharmaceutical composition further comprises a chemosensitizing amount of one or more chemosensitizing agent(s).

Hence, according to yet another aspect of the present invention there is provided a pharmaceutical composition which comprises a chemotherapeutically effective amount of one or more chemotherapeutic agent(s), being entrapped in a drug carrier, a chemosensitizing effective amount of one or more chemosensitizing agent(s), and a chemoprotective effective amount of one or more chemoprotecting agent(s).

The pharmaceutical compositions of the present invention are all identified for use in cancer therapy and hence can be packaged in a container and identified in print on or in the container for use in cancer therapy.

According to further aspects of the present invention there are provided pharmaceutical kits, which comprise, as a chemotherapeutically active ingredient, one or more chemotherapeutic agent(s) being entrapped in a drug carrier, one or more chemosensitizing agent(s) and/or one or more chemoprotecting agent(s). The one or more chemotherapeutic agent(s), chemosensitizing agent(s) and chemoprotecting agent(s) are individually packaged within each of the pharmaceutical kits.

The pharmaceutical kits of the present invention are preferably identified in print for use in cancer therapy.

According to still further aspects of the present invention there are provided methods of treating cancer in a subject in need thereof The methods comprise administering to the subject a chemotherapeutically effective amount of one or more chemotherapeutic agent(s) being entrapped in a drug carrier, and administering to the subject a chemosensitizing effective amount of one or more chemosensitizing agent(s) and/or a chemoprotective effective amount of one or more chemoprotecting agent(s).

For each of the above methods, the administration of each of the chemotherapeutic agent(s), chemosensitizing agent(s) and chemoprotecting agent(s) can be performed prior to, concomitant with or following the administration of the other agent(s).

According to further features in preferred embodiments of the invention described below, the drug carrier is a targeted drug carrier having an affinity to cancer cells.

According to still further features in the described preferred embodiments the drug carrier comprises liposomes, preferably bioadhesive liposomes such as, for example bioadhesive liposomes that comprise a hyaluronic acid.

According to still further features in the described preferred embodiments the chemotherapeutic agent is selected from the group consisting of an alkylating agent, an antimetabolite, a natural product, a miscellaneous agent, a hormone and an antagonist.

According to still further features in the described preferred embodiments the chemotherapeutic agent is doxorubicin or mitomycin C.

According to still further features in the described preferred embodiments the chemosensitizing agent is selected from the group consisting of a calcium channel blocker, a calmodulin inhibitor, an indole alkaloid, a quinolines, a lysosomotropic agent, a steroid, a triparanol analog, a detergent, a cyclic peptide antibiotic, a psychotherapeutic agent, a cyclic psychotropic agent, and a 3-aryloxy-3-phenylpropylamine.

According to still further features in the described preferred embodiments the chemosensitizing agent comprises a calcium channel blocker and the calcium channel blocker is verapamil.

According to still further features in the described preferred embodiments the chemosensitizing agent is Lithium.

According to still further features in the described preferred embodiments the chemoprotecting agent is selected from the group consisting of a bis-dioxopiperazine, a D-methionine, a thiol-containing compound, a selenol-containing compounds an amifostine, an ergotamine, a pyrridoxine, a lymphotoxin, a DPPE analog and a psychotropic agent.

According to still further features in the described preferred embodiments the chemotherapeutic agent is doxorubicin or mitomycin C, which is entrapped in a targeted drug carrier which comprises liposomes. Preferably, the targeted drug carrier comprises bioadhesive liposomes.

According to still farther features in the described preferred embodiments the doxorubicin and/or mitomycin C, entrapped as described hereinabove, are used with Lithium and/or verapamil as the chemosensitizing agent(s).

According to another aspect of the present invention there is provided a method of chemosensitizing cancer cells in a subject in need thereof, the method comprising administering to the subject a chemosensitizing effective amount of Lithium.

According to yet another aspect of the present invention there is provided a method chemosensitizing MDR cancer cells in a subject in need thereof, the method comprising administering to the subject a chemosensitizing effective amount of Lithium.

According to still another aspect of the present invention there is provided a method of treating cancer in a subject in need thereof, the method comprising administering to the subject a chemosensitizing effective amount of Lithium and a chemotherapeutically effective amount of a chemotherapeutic agent.

According to an additional aspect of the present invention there is provided a method of treating MDR cancer in a subject in need thereof, the method comprising administering to the subject a chemosensitizing effective amount of Lithium and a chemotherapeutically effective amount of a chemotherapeutic agent.

According to another aspect of the present invention there is provided a pharmaceutical composition for treating cancer comprising, as a chemosensitizing agent a chemosensitizing effective amount of Lithium and a chemotherapeutically effective amount of a chemotherapeutic agent.

According to yet another aspect of the present invention there is provided a pharmaceutical composition for treating MDR cancer comprising, as a chemosensitizing agent a chemosensitizing effective amount of Lithium and a chemotherapeutically effective amount of a chemotherapeutic agent.

According to still another aspect of the present invention there is provided a pharmaceutical composition for chemosensitizing cancer cells in a subject in need thereof, comprising, as a chemosensitizing agent, a chemosensitizing effective amount of Lithium, the pharmaceutical composition is packaged in a package and is identified in print in or on the package for use in chemosensitizing.

According to yet an additional aspect of the present invention there is provided a pharmaceutical kit comprising, as a chemotherapeutically active ingredient, at least one chemotherapeutic agent, and Lithium, wherein the at least one chemotherapeutic agent and the Lithium are individually packaged within the pharmaceutical kit.

The present invention successfully addresses the shortcomings of the presently known configurations by providing pharmaceutical compositions, methods and pharmaceutical kits for enhancing the therapeutic efficiency of chemotherapeutic drugs. The pharmaceutical compositions, methods and kits of the present invention are highly advantageous in the treatment of cancer and, in particular, in the treatment of MDR cancer cells.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a scheme illustrating an MDR cell under the dynamic in vivo conditions, showing the entry of free chemotherapeutic drug (CT) into the cell by passive diffusion across the cell membrane; the clearance of free drug at the extracellular space outside the cell and the extrusion channel pumping the drug out of the cell

FIG. 2 is a scheme illustrating the combined therapeutic effect of the compositions of the present invention on an MDR cell under the dynamic in vivo conditions, showing a chemosensitizer (CS) inside the cell; the inhibited pump; and the site-adherent site-retained targeted carrier entrapping the chemotherapeutic drug (CT) bound to the cell membrane.

FIG. 3 presents comparative bar graphs demonstrating the effect of various formulations of doxorubicin and a chemosensitizer in cultures of C26 cells, compared with the cytotoxic effect of the chemotherapeutic drug without a chemosensitizer, upon a short-term, 4 hours, exposure. The doxorubicin is in three different formulations: as a free drug (denoted F-DOX), entrapped in regular liposomes (denoted RL-DOX) and entrapped in bioadhesive liposomes that have HA as their bioadhesive ligand (denoted BAL-DOX). The drug dose in all formulations is 0.2 μg/ml. Left-hand side: the chemosensitizer is verapamil, at 15 μM. Right-hind side: the chemosensitizer is Lithium, at 1 μM. Each bar is an average of 32-64 wells and the error bars represent the standard deviations.

FIG. 4 presents comparative bar graphs demonstrating the effect of various formulations of doxorubicin and Lithium as a chemosensitizer in cultures of C6 cells, compared with the cytotoxic effect of the chemotherapeutic drug without Lithium, upon a short-term, 4 hours, exposure (right-hand side) and upon incubation with the therapeutic media for 24 hours. The doxorubicin is in three different formulations: free (denoted F-DOX), entrapped in regular liposomes (denoted RL-DOX) and entrapped in bioadhesive liposomes that have HA as their bioadhesive ligand (denoted BAL-DOX). The drug dose in all formulations is 0.2 μg/ml Each bar is an average of 32-64 wells and the error bars represent the standard deviations.

FIG. 5 presents comparative bar graphs demonstrating the effect of various formulations of mitomycin C (MMC) and Lithium as a chemosensitizer in cultures of B16F10 cells, compared with the cytotoxic effect of the chemotherapeutic drug without Lithium, upon a short-term, 2 hours, exposure. The mitomycin C (MMC) is in three formulations: free (denoted F-MMC), entrapped in regular liposomes (denoted RL-MMC) and entrapped in bioadhesive liposomes that have HA as their bioadhesive ligand (denoted BAL-MMC). The drug dose in all formulations is 50 μg/ml. The Lithium dose is 1 mM. Each bar is an average of 32-64 wells and the error bars represent the standard deviations.

FIG. 6 presents comparative plots demonstrating the effect of various concentrations of Lithium and of 15 μM verapamil as chemosensitizers, on doxorubicin efflux from C6 cells. Each value in the plots is an average of 3-6 wells, with a standard deviations lower than 5%.

FIG. 7 are comparative plots demonstrating the effect 1 mM Lithium as a chemosensitizer, on doxorubicin and MMC efflux from C6 cells. Each value in the plots is an average of 3-6 wells, with a standard deviations lower than 5%.

FIG. 8 presents comparative plots demonstrating the survival of BALB/c mice inoculated with C26 cells to the right-hind foot pad, upon treatment with saline, free MMC, MMC-BAL, Free-MMC and Lithium and MMC-BAL and Lithium. Lithium was given in the drinking water (dose of 1 mM) from the day of tumor inoculation, during the whole experiment period MMC, fee or entrapped, was administered intravenously by injections to the tail vein, at days 5, 12 and 19 from tumor inoculation. Each injection volume was 100 μl.

### DESCRIPTION OF THE PREFERRED EMBODIMENTS

The present invention is of pharmaceutical compositions, methods and pharmaceutical kits which can be efficiently used in cancer therapy. Specifically, the present invention can be used to improve the therapeutic activity of a chemotherapeutic agent in the treatment of cancer, and in particular, in the treatment of MDR cancer cells.

The principles and operation of the pharmaceutical compositions and methods according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

As is discussed in detail in the Background section, drug resistance and in particular multidrug resistance (MDR) is one of the major prevalent problems in cancer therapy. To date, several methods have been developed to enhance the cytotoxicity of chemotherapeutic drugs for MDR treatment, with the aim of increasing the intracellular level of the drug. These methods have been generally directed toward inhibition of the drug efflux from the cell, typically by using chemosensitizers that inhibit the extrusion pumps. These methods are typically limited by the toxicity of the required dose of the chemosensitizers and hence do not provide a substantial improvement of the therapeutic activity of the chemotherapeutic drug. In addition, these treatments, while affecting the efflux of the drug o the cells, do not alter the influx thereof, whereby the final intracellular concentration of the drug depends on the ratio between the influx and efflux.

While conceiving the present invention, it was hypothesized that a chemotherapeutic treatment that is directed toward both decreasing the drug efflux and increasing the drug influx can provide a promising solution for MDR chemotherapy in clinic. The underlying basis for this hypothesis was as follows:

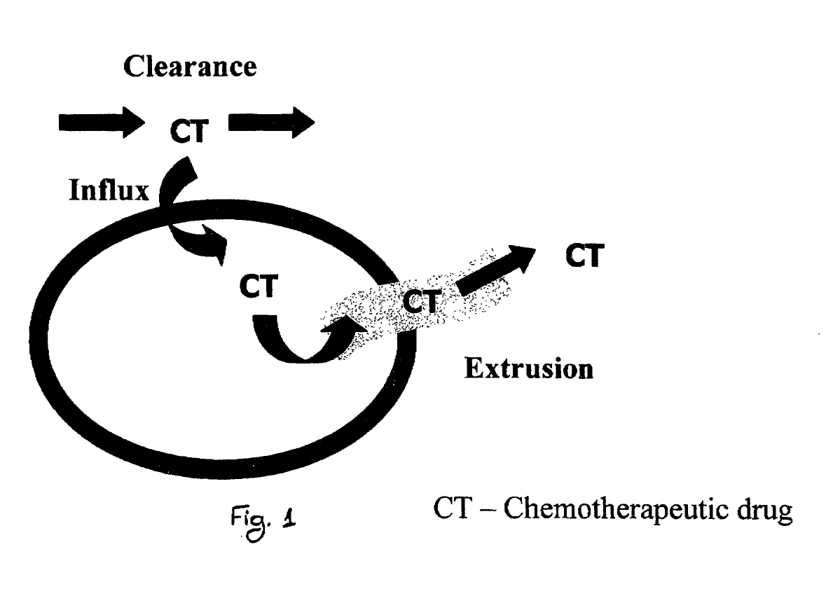

As is discussed in detail hereinabove and is further illustrated in FIG. 1, under the dynamic conditions that prevail in vivo, the accumulation of the administered drug at the extracellular region outside the tumor cell is accompanied by a natural clearance processes that remove the drug from the vicinity of the tumor, which results in a short duration and reduced magnitude of the drug influx and hence in an intracellular level of the drug that is often under its lethal threshold.

In view of the above, it was hypothesized that a combined treatment that is aimed at simultaneously increasing the drug influx and decreasing the drug efflux would result in increased duration and level of the intracellular loading of the chemotherapeutic drug, and hence in enhanced activity of the chemotherapeutic drug.

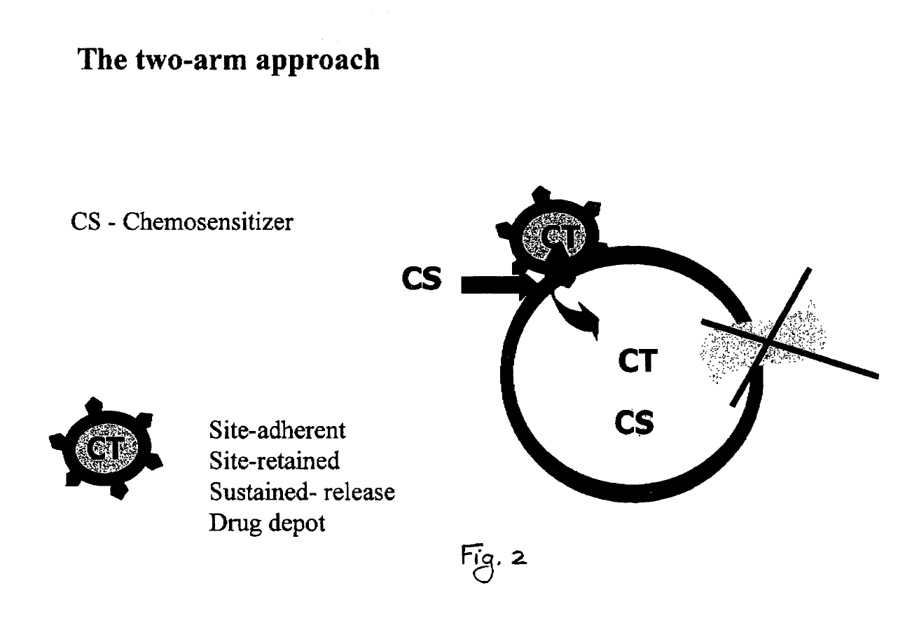

As is schematically illustrated in FIG. 2, this combined treatment is based on a multi-arm approach in which one arm decreases the drug efflux using a chemosensitizer (CS) that inhibits the extrusion pumps, as in the presently known method, and another arm increases the drug influx by encapsulating the chemotherapeutic drug (CT) in a sustained-release drug carrier that is characterized by high affinity to the tumor cell membrane. It was hypothesized that the high affinity of the carrier to a recognition site on the cell membrane would endow it with the ability to be retained at the targeted cell for prolonged time period, even during the undesirable clearance processes, and hence would create a depot of the drug at the cell surface, increasing the magnitude and the duration of the driving force for the drug influx.

Alternatively, the multi-arm approach further includes a chemoprotective agent, as a third arm or as one of the two-arms described hereinabove, replacing the chemosensitizer. It is hypothesized that by reducing, preventing or ameliorating the toxic side effects of the chemotherapeutic drug, the chemoprotective agent would enable the administration of increased dose of the encapsulated drug and hence would lead to increased concentration of the chemotherapeutic drug at the cell surface The increased concentration of the drug would substantial shift the influx/efflux balance, to thereby achieve an enhanced intracellular level of the drug.

Such a combined chemotherapeutic treatment, which addresses both drug influx and drug efflux, has never been practiced hitherto.

While reducing the present invention to practice, as is further described in the Examples section that follows, it was found that this multi-arm, combined treatment resulted in a synergistic effect of the administered agents both in vitro and in vivo and hence in elevating the intracellular drug levels above lethal threshold. This synergistic effect was found highly efficient in overcoming MDR.

Hence, the present invention is of a novel treatment for effecting tumor (both solid and non-solid) chemotherapy, based on the combination of chemotherapeutic drugs that are standard therapy protocols in the clinic such as, but not limited to, doxorubicin and mitomycin C, being entrapped in recognized targeted drug carriers; chemosensitizing agents that can be either well known chemosensitizers or novel compounds that exerts chemosensitizing effect; and/or chemoprotective agents that are standard therapy protocols in the clinic. It is shown herein that this combined treatment leads to significant increases in efficacy of the cytotoxic drugs and is especially effective in tumors that are resistant (MDR) to the chemotherapeutic drugs.

Hence, each of the pharmaceutical compositions of the present invention comprises a chemotherapeutically effective amount of one or more chemotherapeutic agent(s) entrapped in a drug carrier, a chemosensitizing effective amount of one or more chemosensitizing agent(s) and/or a chemoprotective amount of one or more chemoprotecting agent(s). As is discussed hereinabove, enhanced therapeutic activity of the entrapped chemotherapeutic drug can be achieved, according to the present invention, when used in combination with a chemosensitizing agent, a chemoprotecting agent or a combination thereof.

As used herein, the phrase “chemotherapeutically effective amount” describes an amount of a chemotherapeutic agent administered to an individual, which is sufficient to cause inhibition, slowing or arresting of the growth of cancerous cells, or which is sufficient to produce cytotoxic effect on cancerous cells.

As used herein, the phrase “chemosensitizing effective amount” describes an amount of a chemosensitizing agent administered to an individual, in conjunction with a chemotherapeutic agent, which is sufficient to increase the susceptibility of cancer cells to the chemotherapeutic agent or is sufficient to enhance the cytotoxicity of the chemotherapeutic agent.

The phrase “chemoprotective effective amount”, as used herein, describes an amount of a chemoprotecting agent administered to an individual, in conjunction with a chemotherapeutic agent, which is sufficient to protect normal, non-cancer cells from adverse side effects induced by the chemotherapeutic agent or, in other words, is sufficient to reduce, prevent or otherwise ameliorate the adverse side effects of the chemotherapeutic drug on normal cells.

According to the present invention, the chemotherapeutic agent, which is also referred to herein interchangeably as “a chemotherapeutic drug”, “cytotoxic drug”, “anti-cancer drug”, “antineoplastic drug” or simply “a drug”, may be, for example, one of the following: an alkylating agent such as a nitrogen mustard, an ethyleneimine and a methylmelamine, an alkyl sulfonate, a nitrosourea, and a triazene; an antimetabolite such as a folic acid analog, a pyrimidine analog, and a purine analog; a natural product such as a vinca alkaloid, an epipodophyllotoxin, an antibiotic, an enzyme, a taxane, and a biological response modifier; miscellaneous agents such as a platinum coordination complex, an anthracenedione, an anthracycline, a substituted urea, a methyl hydrazine derivative, or an adrenocortical suppressant; or a hormone or an antagonist such as an adrenocorticosteroid, a progestin, an estrogen, an antiestrogen, an androgen, an antiandrogen, or a gouadotropin-releasing hormone analog. Specific examples of allylating agents, antimetabolites, natural products, miscellaneous agents, hormones and antagonists, and the types of cancer for which these classes of chemotherapeutic agents are indicated are provided in Table 1.

A presently preferred chemotherapeutic agent is doxorubicin or mitomycin C. However, it is envisioned by the inventors of the present invention and is further supported by the experimental results presented in the Examples section that follows, that the combined treatment of the present invention can be utilized for enhancing the cytotoxicity of a variety of chemotherapeutic agents having differing mechanisms of action. A listing of currently available chemotherapeutic agents according to class, and including diseases for which the agents are indicated, is provided in Table 1.

TABLE 1
Chemotherapeutic Agents Useful in Neoplastic Disease¹

| Class | Type of Agent | Name | Disease² |
|---|---|---|---|
| Alkylating | Nitrogen | Mechlorethamine | Hodgkin's disease, non-Hodgkin's |
| Agents | Mustards | (HN₂) | lymphomas |
| | | Cyclophosphamide | Acute and chronic lymphocytic |
| | | Ifosfamide | leukemias, Hodgkin's disease, |
| | | | non-Modgkin's lymphomaa, |
| | | | neuroblastoma, breast, Ovary, |
| | | | lung, Wilms' tumor, cervix, |
| | | | testis, soft-tissue sarcomas |
| | | [symbol] lphalan | Multiple myeloma, breast, ovary |
| | | [symbol] lorambucil | Chronic lymphocytic leukemia, |
| | | | primary macroglobulinemia, |
| | | | Hodgkin's disease, non- |
| | | | Hodgkin's lymphomas |
| | | Estramustine | Prostate |
| | Ethylenimines | Mexamethyl- | Ovary |
| | And | melamina | |
| | Methylmelamines | Thiotepa | Bladder, breast, ovary |
| | Alkyl | Busulfan | Chronic granulocytic leukemia |
| | Sulfonates | | |
| | Nitrosoureas | Carmustine | Hodgkin's disease, non-Hodgkin's |
| | | | lymphoinas, primary brain |
| | | | tumora, multiple myeloma, |
| | | | malignant melanoma |
| | | Lomustine | Hodgkin's disease, non-Hodgkin's |
| | | | lymphomas, primary brain |
| | | | tumors, small-cell lung |
| | | Semustine | Primary brain tumors, stomach, |
| | | | colon |
| | | Streptozocin | Malignant pancreatic insulinoma, |
| | | | malignant carcinoid |
| | Triazenes | Dacarbazine | Malignant melanoma, Hodgkin's |
| | | Procarbazine | disease, soft-tissue sarcomas |
| | | Aziridine | |
| Antimetabolites | Folic Acid | Methotrexate | lymphocylic leukemia, |
| | Analogs | Trimetrexate | choriocarcinozsa, mycosis |
| | | | fungoides, breast, head and |
| | | | neck, lung, osteogenic sarcoma |
| | Pyrimidine | Fluorouracil | Breast, colon, stomach, pancreas, |
| | Analogs | Floxuridine | ovary, head and neck, urinary |
| | | | bladder, prezoalignant skin |
| | | | lesions (topical) |
| | | Cytarabine | Acute granulocytic and acute |
| | Purine Analogs | Azacitidine | lymphocytic leukemias |
| | and Related | Mercaptopurine | lymphocytic, acute |
| | Inhibitors | | granulocytic, and chronic |
| | | | granulocytic leukemias |
| | | Thioguanine | Acute granulocytic, acute |
| | | | lymphocytic, and chronic |
| | | | granulocytic leukemias |
| | | Pentostatin | Hairy cell leukemia, mycosis |
| | | | fungoides, chronic lymphocytic |
| | | | leukemia |
| | | Fludarabine | Chronic lymphocytic leukemia, |
| | | | Hodgkin's and non-Hodgkin's |
| | | | lymphomas, mycosis fungoides |
| Natural | Vinca Alkaloids | Vinblastine (VLB) | Hodgkin's disease, non-Hodgkin's |
| Products | | | lymphomas, breast, testis |
| | | Vincristine | Acute lymphocytic leukemia, |
| | | | neuroblastoma, Wilms' tumor, |
| | | | rhabdomyosarcona, Hodgkin's |
| | | | diaease, non-Hodgkin's |
| | | | lymphomas, small-cell lung |
| | | Vindesine | Vinta-resistant acute lymphocytic |
| | | | leukemia, chronic myelocytic |
| | | | leukemia, melanoma, lymphomas, |
| | | | breast |
| | Epipndophyl- | Etoposide | Testis, small-cell lung and other |
| | Lotoxins | Teniposide | lung, breast, Hodgkin's |
| | | | disease, non-Hodgkin's |
| | | | lymphomas, acute granulocytic |
| | | | leukemia, Kaposi's sarcoma |
| | Antibiotics | Dactanomycin | Choriocarcinoma, Wilms' tumor. |
| | | | rhabdomyosarcoma testis, |
| | | | Kaposi's sarcoma |
| | | Daunorubicin | Acute granulocytic and acute |
| | | | lymphocytic leukemias |
| | | Doxorilbicin | Soft-tissue, osteogenic, and |
| | | 4′- | other sarcomas; Hodgkin's |
| | | Deoxydoxorubicin | disease. rice-Hodgkin's |
| | | | lymphomas, acute leukemias, |
| | | | breast, genitourinary, thyroid, |
| | | | lung, stomach, neuroblastoma |
| | | Bleomycin | Testis, head and neck, skin, |
| | | | esophagus, lunq, and |
| | | | genitourinary tract: |
| | | | Hodgkin's disease, non- |
| | | | Hodgkin's lymphonas |
| | | Placamycin | Testis, malignant hypercalcemia |
| | | Mitomycin | Stomach, cervix. colon, breast, |
| | | | pancreas, bladder, head and |
| | | | neck |
| | Enzymes | Asparaginase | Acute lymphocytic leukemia |
| | Taxanes | Docetaxel | Breast, ovarian |
| | | Paclitaxel | |
| | Biological | Interferon Alfa | Hairy cell leukemia, Kaposi's |
| | Response | | sarcoma, melanoma, carcinoid, |
| | Modifiers | | cell, ovary, bladder, |
| | | | non-Hodgkin's lymphomas, |
| | | | mycosis fungoides, multiple |
| | | | myeloma, chronic granulucytic |
| | | | leukemia |
| | | Tumor | Investigational |
| | | Fractor | |
| | | Tumor- | Investigational |
| | | Infiltrating | |
| | | Lymphocytes | |
| Miscellaneous | Platinum | Cisplatin | Testis, ovary, bladder, head and |
| Agents | Coordination | Carboplatin | neck, lung, thyroid, cervix, |
| | Complexes | | endometrium, neuroblastoma, |
| | | | osteogenic Sarcoma |
| | Anthracenedione | Mitoxantrone | Acute granulocytic leukemia, |
| | | | breast |
| | Substituted | Hydroxyurea | Chronic granulocytic leukemia, |
| | Urea | | polycythemia vera, essential |
| | | | thrombocytosis, malignant |
| | | | melanoma |
| | Methyl | Procarbazine | Hodgkin's disease |
| | Hydrazine | | |
| | Derivative | | |
| | Adrenocortical | Mitotane | Adrenal cortex |
| | Suppressant | Aminoglutethimide | Breast |
| Hormonea and | | | Acute and chronic lymphocytic |
| Antagonists | costeroids | | leukemias, non-Hodgkin's |
| | | | lymphomas, Hodgkin's disease, |
| | | | breast |
| | Progestins | Hydroxy- | Endometrium, breast |
| | | progesterone | |
| | | caproate | |
| | | Medroxy- | |
| | | progesterone | |
| | | acetate | |
| | | Megestrol acetate | |
| | Estrogens | Diethylstil- | Breast, prostate |
| | | bestrol | |
| | | Ethinyl estradiol | |
| | Antiestrogen | Tamoxifen | |
| | Androgens | tosterone | |
| | | propionate | |
| | | Fluoxymesterone | |
| | Antiandrogen | flutamide | Prostate |
| | Gonadotropin- | Leuprolide | Prostate, Estrogen-receptor- |
| | Releasing | Goserelin | positive brest |
| | hormone | | |
| | analog | | |
| ¹Adapted from Calabresi, P., and B. A. Chabner, “Chemotherapy of Neoplastic Diseases” Section XII, pp 1202-1263 in: Goodman and Gilman's The Pharmacological Basis of Therapeutics, Eighth ed., 1990 Pergamin Press, Inc.; and Barrows, L. R., “Antineoplastic and immunoactive Drugs”, Chapter 75, pp 1236-1262, in: Remington: The Science and Practice of Pharmacy, Mack Publishing Co. Easton, PA, 1995.; both references are incorporated by reference herein, in particular for treament | | | |
| # protocols. | | | |

²Neoplasms are carcinomas unless otherwise indicated

According to the present invention, the chemotherapeutic drug is entrapped in a drug carrier, which is aimed at providing for site-directing, site-adherence, site-retain and sustained release of the chemotherapeutic drug.

Drug carriers which are usable for entrapping a chemotherapeutic agent, according to the present invention, include any compound, matrix and system that is biodegradable and biocompatible. Representative examples of suitable drug carriers include, without limitation, particulate systems, non-particulate systems and sol-gel matrices.

Representative examples of suitable particulate systems include, without limitation, cells, microspheres, nanospheres, viral envelopes, liposomes and other lipid or detergent based particles.

Representative examples of suitable non-particulate systems include, without limitation, proteins and synthetic or bioavailable polymers such as, but not limited to, polysaccharides.

As is discussed hereinabove, the drug carrier entrapping the chemotherapeutic drug should be site-directed, site-adherent and site-retained carrier.

As used herein, the terms “site-directed” and “site-directing” means having specificity for targeted sites. “Specificity for targeted sites” means that upon contacting the site-directed carrier with the targeted site (e.g., cancer cells), for example, under physiological conditions of ionic strength, temperature, pH and the like, specific binding will occur. The interaction may occur due to specific electrostatic, hydrophobic, entropic or other interaction of certain residues of the carrier with specific residues of the target to form a stable complex under conditions effective to promote the interaction.

The terms “site-adherent”, “site-adhering”, “bioadhesive” “site-retained” and “site-retain” are used herein to describe the formation of the stable complex described hereinabove between the carrier and the targeted site.

Hence, preferred drug carriers, according the present invention, are targeted drug carriers that have an affinity to cancer cells.

The phrase targeted drug carriers that have an affinity to cancer cells” is used herein to describe drug carriers that upon contacting with the targeted site (e.g., cancer cells), for example, under physiological conditions of ionic strength, temperature, pH and the like, specifically bind thereto. The interaction may occur due to specific electrostatic, hydrophobic, entropic or other interaction of certain residues of the carrier with specific residues of the target to form a stable complex under conditions effective to promote the interaction.

The phrase “affinity to cancer cells” means that the drug carrier remains adhered to the surface of the cancer cells for prolonged time period. Alternatively, the targeted drug carrier can be internalized into the cell and be retained therein.

Preferred targeted drug carriers, according to the present invention, include liposomes.

Liposomes have been extensively studied as drug carriers and offer a range of advantages relative to other drug carriers. Composed of naturally occurring materials which are biocompatible and biodegradable, liposomes are used to encapsulate biologically active materials for a variety of purposes. Having a variety of layers, sizes, surface charges and compositions, numerous procedures for liposomal preparation and for drug encapsulation therewithin have been developed, some of which have been scaled up to industrial levels. Liposomes can be designed to act as sustained release drug depots and, in certain applications, aid drug access across cell membranes.

As liposomes are typically characterized by limited target abilities, limited retention and stability in circulation, potential toxicity upon chronic administration and inability to extravasate, many successful works have been made to bind different substances to liposomes. For example, recognizing substances, including antibodies, glycoproteins and lectins, have been bound to liposomal surfaces in an attempt to confer target specificity to the liposomes.

U.S. Pat. Nos. 5,401,511, 5,603,872 and 5,846,561, which are incorporated by reference as if fully set forth herein, disclose efficient methodologies to effectively bind various recognizing substances such as, but not limited to, collagen, gelatin, hyaluronic acid and epidermal growth factor, to liposomal surfaces. The phrase “recognizing substances” means “site-directing” substances, as this term is defined hereinabove.

Hence, preferred targeted drug carriers according to the present invention include liposomes that bind one or more site-directing substances.

Site-directing substances that are suitable for use in the context of the present invention include, without limitation, collagen, gelatin, hyaluronic acid, epidermal growth factor, antibodies, folic acid, transferin, LDL and lectins.

Preferred liposomes according to the present invention are liposomes that bind one or more site-directing substances and are characterized as bioadhesive liposomes.

The phrase “bioadhesive liposomes” is used herein to describe liposomes that bind recognizing substances which are able to adhere or glue to the designated target cells, and hence retain the modified liposomes onto a target cells despite cellular and fluid dynamics for sustained release of the liposomes therapeutic contents.

The presently preferred bioadhesive liposomes include liposomes that bind hyaluronic acid (HA).

Hence, the pharmaceuticals compositions of the present invention preferably include, as one arm of the multi-arm composition, a chemotherapeutic agents such as doxorubicin or mitomycin C, entrapped in targeted liposomes, preferably bioadhesive liposomes such as liposomes that bind hyaluronic acid.

Additional arms of the multi-arm pharmaceutical compositions of the present invention include a chemosensitizing agent and/or a chemoprotective agents.

According to the present invention, the chemosensitizing agent can be any compound or mixture of compounds that cause chemosensitizing effect.

The phrase “chemosensitizing agent” and “chemosensitizing effect” are used herein to describe an agent and an effect, respectively, that render a cancer cell susceptible to a chemotherapeutic agent or that enhance the cytotoxicity of a chemotherapeutic agent.

Representative examples of chemosensitizing agents include, without limitation, calcium channel blockers such as verapamil, calmodulin inhibitors such as trifluoperazine, indole alkaloids such as reserpine, quinolines such as quinine, lysosomotropic agents such as chloroquine, steroids such as progesterone), triparanol analogs such as tamoxifen, detergents such as cremophor EL, cyclic peptide antibiotics such as cyclosporines, cyclic psychotropic agents such as antidepressants and phenothiazines, 3-aryloxy-3-phenylpropylamines such as fluxetine, lymphotoxins, psychotherapeutic agents and derivatives thereof.

A preferred chemosensitizing agent, according to the present invention is verapamil, which is known for its activity as a calcium channel blocker. As is discussed hereinabove, the use of verapamil as a chemosensitizer has been practiced. However, the verapamil dose required for effective MDR reversal is high above its toxic domain and hence the use thereof in the presently known chemotherapy methods is limited.

While reducing the present invention to practice, it was surprisingly found that Lithium, which is known as a psychotherapeutic drug that is used in the treatment of severe mania-dipressia, exerts efficient chemosensitizing effect when administered in combination with a chemotherapeutic drug.

Hence, according to an aspect of the present invention there is provided a method chemosensitizing cancer cells in a subject in need thereof, the method comprising administering to the subject a chemosensitizing effective amount of Lithium.

According to another aspect of the present invention there is provided a method of chemosensitizing MDR cancer cells in a subject in need thereof, the method comprising administering to the subject a chemosensitizing effective amount of Lithium.

According to still another aspect of the present invention there is provided a method of treating cancer in a subject in need thereof, the method comprising administering to the subject a chemosensitizing effective amount of Lithium and a chemotherapeutically effective amount of a chemotherapeutic agent.

According to an additional aspect of the present invention there is provided a method of treating MDR cancer in a subject in need thereof, the method comprising administering to the subject a chemosensitizing effective amount of Lithium and a chemotherapeutically effective amount of a chemotherapeutic agent.

According to another aspect of the present invention there is provided a pharmaceutical composition for treating cancer comprising, as a chemosensitizing agent, a chemosensitizing effective amount of Lithium and a chemotherapeutically effective amount of a chemotherapeutic agent.

According to yet another aspect of the present invention there is provided a pharmaceutical composition for treating MDR cancer comprising, as a chemosensitizing agent a chemosensitizing effective amount of Lithium and a chemotherapeutically effective amount of a chemotherapeutic agent.

According to still another aspect of the present invention there is provided a pharmaceutical composition for chemosensitizing cancer cells in a subject in need thereof, comprising, as a chemosensitizing agent, a chemosensitizing effective amount of Lithium, the pharmaceutical composition is packaged in a package and is identified in print in or on the package for use in chemosensitizing.

According to yet an additional aspect of the present invention there is provided a pharmaceutical kit comprising, as a chemotherapeutically active ingredient, at least one chemotherapeutic agent, and Lithium, wherein the at least one chemotherapeutic agent and the Lithium are individually packaged within the pharmaceutical kit.

Thus, the presently most preferred chemosensitizing agent according to the present invention is Lithium. As is well demonstrated in the Examples section that follows, a combined treatment of doxorubicin or mitomycin C, entrapped in bioadhesive liposomes, and Lithium provides for synergistic effect in enhancing the therapeutic activity of the entrapped chemotherapeutic drug, using a Lithium dose that is well within its toxic domain. The Lithium is typically used in the pharmaceutical compositions of the present invention as is dissolved alkaline salt and is typically administered orally.

Further according to the present invention, the chemoprotecting agent can be any compound or mixture of compounds that cause chemoprotective effect.

The phrases “chemoprotecting agent” and “chemoprotecting effect” are used herein to describe an agent and an effect, respectively, that protect normal cells from adverse side effects induced by a chemotherapeutic agent.

Representative examples of chemoprotective agents include, without limitation, bis-dioxopiperazine compounds, D-methionine, thiol- and selenol-containing compounds such as cysteine, cysteamine, glutathione, selenocysteine, selenocysteamine and NR compounds, amifostines, ergotamines, pyrridoxines, lymphotoxins, DPPE (N,N-diethyl-2-[4-(phenylmethyl)-phenoxyl-ethenamine.HCl) analogs and cyclic psychotropic agents.

As is discussed hereinabove, some of the agents described hereinabove exert both chemosensitizing effect and chemoprotective effects. Limphotoxins are a representative example of such agents. Using these agents in the combined treatment of the present invention provides for a triple effect of chemotherapeutic cytotoxicity, chemosensitization and chemoprotection, of a pharmaceutical composition that comprises only two active ingredients.

The pharmaceutical compositions of the present invention may further comprise a pharmaceutically accepted excipient.

The phrase “pharmaceutically acceptable excipient”, as used herein, describes an inert substance added to a pharmaceutical composition to further facilitate administration of a compound. Examples, without limitation, of excipients include calcium carbonate, calcium phosphate, various sugars and types of starch, cellulose derivatives, gelatine, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration of the different agents of pharmaceutical compositions of the present invention may, for example, include oral, rectal, transmucosal, intestinal or parenteral delivery, including intramuscular, subcutaneous and intramedullary injections as well as intrathecal, direct intraventricular, intravenous, intraperitoneal, intranasal, or intraocular injections.

Pharmaceutical compositions for use in accordance with the present invention thus may be formulated in conventional manner using one or more physiologically acceptable excipients, which facilitate processing of the active agents into preparations which, can be used pharmaceutically. Proper formulation is dependent upon the route of administration chosen.

For injection, the agents of the invention may be formulated in aqueous solutions, preferably in physiologically compatible buffers such as Hank's solution, Ringer's solution, or physiological saline buffer.

For transmucosal administration, penetrants appropriate to the barrier to be permeated are used in the formulation. Such penetrants arc generally known in the art.

For oral administration, the agents of the present invention can be formulated readily by combining same with pharmaceutically acceptable excipients well known in the an. Such excipients enable the active agents to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatine, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active agents doses.

Pharmaceutical compositions, which can be used orally, include push-fit capsules made of gelatine as well as soft, sealed capsules made of gelatine and a plasticizer, such as glycerol or sorbitol. The push-fit capsules may contain the active agents in admixture with filler such as lactose, binders such as starches, lubricants such as talc or magnesium stearate and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by inhalation, the agents for use according to the present invention are conveniently delivered in the form of an aerosol spray presentation from a pressurized pack or a nebulizer with the use of a suitable propellant, e.g., dichlorodifluoromethane, trichlorofluoromethane, dichlorotetrafluoroethane or carbon dioxide. In the case of a pressurized aerosol, the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

The compositions described herein may be formulated for parenteral administration, e.g., by bolus injection or continuos infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active agents in water-soluble form. Additionally, suspensions of the active agents may be prepared as appropriate oily injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the agents to allow for the preparation of highly concentrated solutions.

Alternatively, the active agent may be in powder form for constitution with a suitable vehicle, e.g., sterile, pyrogen-free water, before use.

The compositions of the present invention may also be formulated in rectal compositions such as suppositories or retention enemas, using, e.g., conventional suppository bases such as cocoa butter or other glycerides.

The pharmaceutical compositions herein described may also comprise suitable solid of gel phase excipients. Examples of such excipients include, but are not limited to, calcium carbonate, calcium phosphate, various sugars, starches, cellulose derivatives, gelatin and polymers such as polyethylene glycols.

Many of the active agents in the compositions of the present invention, particularly many of the chemosensitizing agents and the chemoprotecting agents of the present invention, may be provided as physiologically acceptable salts wherein the agent may form the negatively or the positively charged species. Examples of salts in which the agents form the positively charged moiety include, without limitation, quaternary ammonium, salts such as the hydrochloride, sulfate, carbonate, lactate, tartrate, malcate, succinate, etc., wherein the nitrogen of the quaternary ammonium group is a nitrogen of a compound of the present invention which reacts with an appropriate acid. Salts in which the agents form the negatively charged species include, without limitation, the sodium, potassium, calcium and magnesium salts formed by the reaction of a carboxylic acid group in the molecule with the appropriate base (e.g., sodium hydroxide (NaOH), potassium hydroxide (KOH), calcium hydroxide (Ca(OH)₂), etc.).

Pharmaceutical compositions suitable for use in context of the present invention include compositions wherein the active agents are contained in an amount effective to achieve the intended purpose.

Determination of the effective amount of each of the agents of the present invention is well within the capability of those skilled in the art, especially in light of the detailed disclosure provided herein.

The effective amount or dose of the agents contained in the pharmaceutical compositions of the present invention can be estimated initially from cell culture assays. For example, a dose can be formulated in animal models to achieve a circulating concentration range that includes the IC₅₀ as determined in cell culture (e.g., the concentration of the test agents, which achieves a half-maximal death of cancer cells). Such information can be used to more accurately determine useful doses in humans.

Toxicity and efficacy of the agents used in context of the present invention can be determined by standard pharmaceutical procedures in cell cultures or experimental animals, e.g., by determining the IC₅₀ and the LD₅₀ (lethal dose causing death in 50% of the tested animals) for a subject compound. The data obtained from these cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p.1).

The amount of a composition to be administered will, of course, be dependent on the subject being treated, the severity of the anticipated affliction, the manner of administration, the judgement of the prescribing physician, etc.

The pharmaceutical compositions of the present invention can be presented in a pack or dispenser device, such as a FDA approved kit, which may contain one or more unit dosage forms containing the active agents. The package is preferably identified in print on or in said container for use in cancer therapy.

The package may, for example, comprise metal or plastic foil, such as a blister package. The package or dispenser device may be accompanied by instructions for administration. The package or dispenser may also be accompanied by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert.

Hence, further according to the present invention, there are provided pharmaceutical kits. Each of the pharmaceutical kits comprises, as a chemotherapeutically active ingredient, one or more chemotherapeutic agent(s) being entrapped in a drug carrier, as is detailed hereinabove, one or more chemosensitizing agent(s) and/or one or more chemoprotecting agent(s), as is further detailed and defined hereinabove. The one or more chemotherapeutic agent(s), chemosensitizing agent(s) and/or chemoprotecting agent(s) are individually packaged within the pharmaceutical kits, in order to avoid interactions therebetween prior to the administration procedure.

The pharmaceutical kits of the present invention are preferably identified in print for use in cancer therapy.

Further according to the present invention, there are provided methods of treating cancer in subjects in need thereof. The methods of the present invention are based on the synergistic effect exerted as a result of the combined administration of one or more chemotherapeutic agent(s), one or more chemosensitizing agent(s) and/or one or more chemoprotective agent(s).

In one method, the cancer treatment is effected by administering to the subject a chemotherapeutically effective amount of one or more chemotherapeutic agent(s), entrapped in a drug carrier, and administering to the subject a chemosensitizing effective amount of one or more chemosensitizing agent(s), prior to, concomitant with or following the administration of the entrapped chemotherapeutic agent(s).

As it is preferable that the chemotherapeutic agent and the chemosensitizing agent exert their activities simultaneously, in order to decrease the drug efflux and increase the drug influx at the same time, thus leading to high level and duration of the intracellular loading of the drug, it is recommended that both agents would be present in the tumor cells at the same time. Hence, the administration of the chemosensitizing agent is preferably performed concomitant with the administration of the chemotherapeutic agent.

In another method, the cancer treatment is effected by administering to the subject a chemotherapeutically effective amount of one or more chemotherapeutic agent(s), entrapped in a drug carrier, and administering to the subject a chemoprotecting effective amount of one or more chemoprotective agent(s), prior to, concomitant with or following the administration of the entrapped chemotherapeutic agent(s).

In yet another method, the cancer treatment is effected by administering to the subject a chemotherapeutically effective amount of one or more chemotherapeutic agent(s), entrapped in a drug carrier, administering to the subject a chemoprotecting effective amount of one or more chemoprotective agent(s), and administering to the subject a chemosensitizing effective amount of one or more chemosensitizing agent(s). The administration of each of the chemotherapeutic agent(s), chemosensitizing agent(s) and chemoprotecting agent(s) in this method can be performed prior to, concomitant with or following the administration of the other agent(s). However, with respect to the administration of the chemosensitizing agent, it is recommended that it is performed concomitant with the administration of the chemotherapeutic drug, as is discussed hereinabove.

The agents that are used in the methods of the present invention include the chemotherapeutic agents, the chemosensitizing agents and the chemoprotecting agents described in detail hereinabove. The effective amounts of the agents are as defined hereinabove.

As used herein, the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “treating” includes abrogating, substantially inhibiting, slowing or reversing the progression of a disease, e.g., cancer, substantially ameliorating clinical symptoms of a disease or substantially preventing the appearance of clinical symptoms of a disease. More specifically, the term “treating” includes substantially inhibiting, slowing or reversing the proliferation of cancer cells or causing death of cancer cells.

The term “cancer” includes various types of malignant neoplasms.

As the methods of the present invention are directed to enhancing the therapeutic activity, and hence the cytotoxicity, of chemotherapeutic drugs, these methods are particularly useful in the treatment of MDR cancer cells.

Hence, the methods of the present invention are particularly useful in the treatment of colon cancer, renal cancer, acute nonlymphocytic leukemia, malignant lymphomas, pancreatic cancer, breast cancer, glioblastoma, rectal cancer and prostate cancer.

It is expected that during the life of this patent many relevant drug carriers, chemotherapeutic agents, chemosensitizing agents and chemoprotective agents will be developed and the scope of these terms is intended to include all such new agents a priori.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Reference is now made to the following examples, which together with the above descriptions, illustrate the invention in a non limiting fashion.

Materials and Experimental Methods

In Vitro Studies:

Chemotherapeutic Agents (CT):

Mitomycin C (MMC) and Doxorubicin (DOX).

Chemosensitizers (CS):

Lithium and Verapamil

Drug Carrier:

Unilamellar regular liposomes (RL) and bioadhesive liposomes bearing hyaluronic acid (HA) as a bioadhesive ligand (HA-BAL), were prepared essentially as described in Margalit et al. J. Cont. Rel. 19:275-288, 1992; Yerusbalmi et al. Biochim. Biophys. Acta., 1189:13-20, 1994; Margalit R. Crit. Rev. in Therapeut. Drug Carr. Sys., 12:233-261, 1995; Yerushalmi et. Al. Arch Biochem. Biophys., 349: 21-26, 1998; Peer et al., Proc. 29^{th} Intl. Symp. Cont. Rel. Bioac. Mater. 2002, which are incorporated by reference as if fully set forth herein.

Cell Lines:

C26 (murine colon adenocarcinoma), C6 (rat glioblastoma) and B16F10 (murine melanoma).

Cell Culture Growth and Maintenance Media:

Dulbecco's modified Eagle's medium (DWEM) for B16F10 and C6 cell lines and RPMI 1640 medium for C26 cells, both supplemented with 10% fetal calf serum (FCS), Penicillin (10,000 units/ml), Streptomycin (10 mg/ml) and L-Glutamine (200 mM) Cell cultures:

Cells were grown in monolayers in 100×20 mm dishes, in the growth media listed above, at 37° C. in 5% CO₂.

Cell Survival:

Cells were grown in monolayers as describe above and seeded onto 96 multiwell plates at a density of 1×10⁴ cells/ml, 24 hours prior to an experiment. Twenty four hours later, the media was replaced by treatment media as is detailed in the Experimental Results section that follows. The experiments were terminated 20-22 hours post media replacement. The quantity of viable cells was determined by the MTT test, recording the absorbencies in a plate reader, at two wavelengths: 550 nm and 650 nm.

Drug Efflux:

Cells were grown in monolayers as described above. Several days prior to an experiment the cells (at a density in the range of 5×10⁴-5×10⁵ cells/ml) were seeded into 6 multiwell culture plates. The experiments were performed when the cells reached confluency.

The efflux experiments were conducted according to the following protocol: Cells of a selected line were incubated overnight with either a non-lethal dose of free drug or the same drug dose combined with a selected dose of Lithium or verapamil. At the end of the incubation the media was removed and the cells were incubated with “drug free media”, which consisted of either buffer alone, for cells loaded with drug only, or buffer that contained the chemosensitizer used at the same dose as in the incubation, for the cells loaded with the drug and the chemosensitizer. This replacement of the loading with the “drug free media” was performed in order to create the driving force for drug efflux from the cell into that external media. At designated periods (time points), the external media was collected and thereafter replaced by fresh “drug free media”, in order to maintain a unidirectional flux, from the cell out. The drug concentration was measured in each of the collected media, and in the cells at the end of the experiment. These data served to calculate the cumulative drug that diffused out of the cells at each time point and to normalize the concentration with respect to the total drug in the system at time=0.

In Vivo Studies:

Animals:

BALB/c mice, female, 8 weeks old at the initiation of experiments were tested. Experiments were performed with 35 mice that were divided into 5 groups. Each group received a specific treatment, as is listed in Table 2 below.

Chemotherapeutic Drug (CT):

Mitomycin C (MMC), injection dose of 10 mg/Kg body.

Drug Carrier:

Bioadhesive liposomes bearing hyaluronic acid (HA) as a bioadhesive ligand (HA-BAL), were prepared as described above for the in vitro experiments.

Chemosensitizer:

Lithium (a 1 mM dose)

Tumor Cells:

C26 cells (originating from mouse colon carcinoma) were grown in cell culture flask essentially as described above under the in vitro experiments. At day 0 of the experiment, the cells were harvested, washed several times with PBS and at the final wash with Hank's buffer, counted and immediately injected subcutaneously into the right-hind foot pad of the mice. The injected dose was 5×10⁵ cells/30 μl.

Treatments and Schedules:

Table 2 below presents the administered treatment and the animals number of each group.

TABLE 2

| Group No. | Treatment | Animals/group |
|---|---|---|
| 1 | Saline | 10 |
| 2 | Free MMC | 10 |
| 3 | Free MMC + Lithium | 5 |
| 4 | MMC in HA-BAL | 5 |
| 5 | MMC in HA-BAL + | 5 |
| | Lithium | |

Saline and the chemotherapeutic formulations were administered on days 5, 12 and 19 of the experiment, by injection into the tail vein. All the injected volumes were 0.1 ml. Lithium, at 1 mM, was given in the drinking water continuously throughout the experiment, from the day of tumor inoculation.

Experimental Results

In Vitro Studies:

Cytotoxicity:

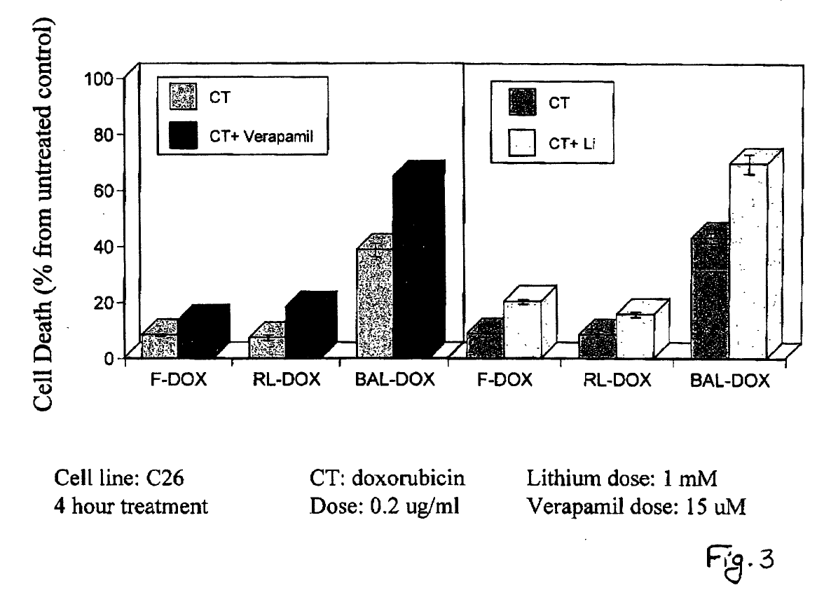

The cytotoxicity studies of the combined treatment of the present invention were conducted in 3 cell lines: C6, C26 and B16F10, as described hereinabove. The obtained results are presented in FIGS. 3-5.

FIG. 3 shows the results obtained upon various treatments in C26 cells, using the classical well established chemosensitizer verapamil in a dual capacity: for itself as a MDR reversal agent, and as a bench mark for the activity of Lithium as a chemosensitizer. The dose selected for verapamil, 15 μM, was the typical dose used in numerous in vitro studies with this agent, although this dose is toxic in vivo. The dose selected for Lithium, 1 mM, was a dose within the in vivo safe range.

In order to decrease the in vitro free-drug bias (as is discussed in detail hereinabove), the selected cell exposure to the treatment media was 4 hours.

FIG. 3 presents the results obtained upon treatments with doxorubicin, with and without a chemosensitizer, in cultures of C26 cells.

The results obtained upon treatments with doxorubicin, with and without verapamil, are presented on the left-hand side of FIG. 3. Doxorubicin was administered as a free chemotherapeutic agent (F-DOX), entrapped in regular liposomes (RL-DOX) and entrapped in bioadhesive liposomes (BAL-DOX), which are also referred to herein as a targeted carrier. As was expected for MDR cells, the free drug and the drug entrapped in regular liposomes had little impact on cell death. Adding verapamil to each of these formulations generated only a slight increase in cell death. In contrast, when the treatment comprised the doxorubicin entrapped in the targeted carrier, without chemosensitization, the cytotoxicity of the drug was increased to an appreciable level, which was further enhanced when the combined treatment with verapamil was applied.

The results obtained upon treatments with doxorubicin, with and without Lithium as a chemosensitizer, are presented on the right-hand side of FIG. 3. Similar effects were observed.

For each of the two chemosensitizers tested, the results of the combined treatment were highly significant statistically, compared to the free drug (P<0.001).

These data confirm two key features: that Lithium acts as an efficient chemosensitizer; and that the combined treatment of the present invention is not limited to any specific chemosensitizer.

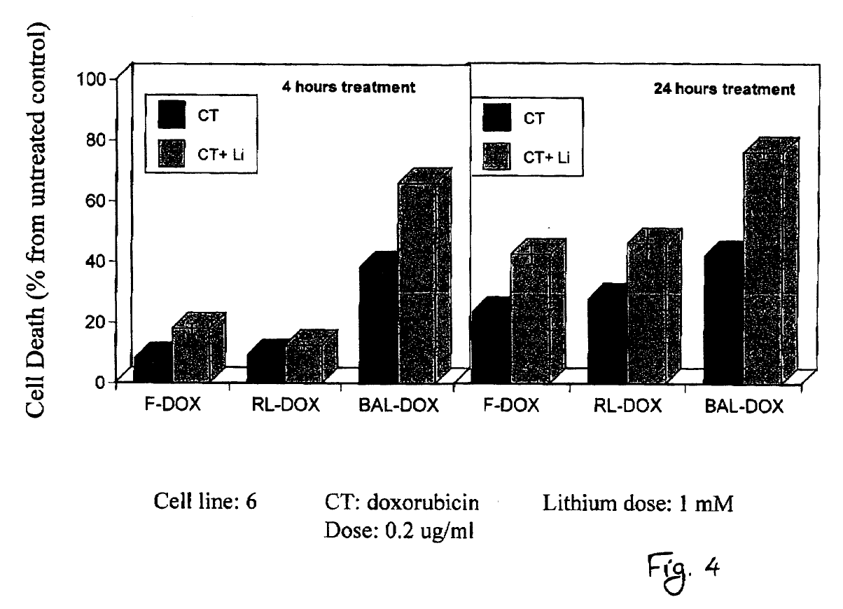

FIG. 4 presents the results obtained upon treatments with doxorubicin, with and without Lithium, in cultures of C6 cells, during different exposure periods.

The results obtained upon 4 hours treatments with doxorubicin, with and without Lithium, are shown in the left-hand side of FIG. 4. The obtained results indicate that the combined approach as well as the performance of Lithium as a chemosensitizer, are not restricted to a single cell line.

In order to demonstrate the bias towards free drug, as well as towards regular, non-targeted, drug carriers, which is present in typical, long-term exposures in vitro, as is discussed hereinabove, identical experiments were conducted for an exposure time of 24 hours. The obtained results are shown in the right-hand side of FIG. 4, and clearly indicate this bias. Nevertheless, the obtained results further demonstrate the efficacy of the combined treatments of the present invention, since even under these bias conditions, the superior cytotoxic activity of the combined treatment is quite evident.

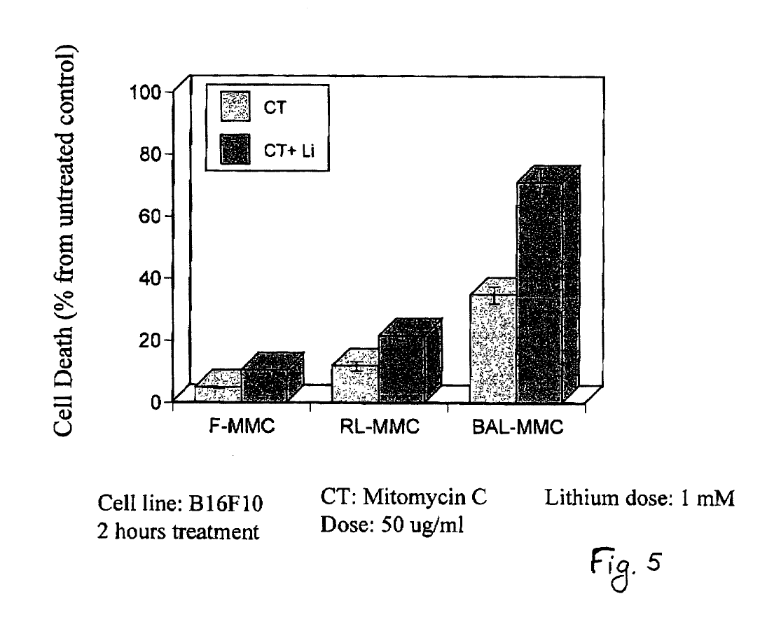

FIG. 5 presents the results obtained for treatments with MMC, with and without Lithium as a chemosensitizer, in cultures of B16F10 cells, during a short-term exposure of 2 hours. Also in this case, the superior cytotoxic activity of the combined treatment of the present invention, as compared with all the other tested treatments, is clearly demonstrated. These results indicate that the enhanced efficacy of the combined treatment of the present invention is not restricted to a single chemotherapeutic drug, and therefore can be practiced with various chemotherapeutic drugs. The obtained results further provide an additional support that this treatment is not restricted to a specific cell line.

Hence, the in vitro data provide ample and substantial experimental support for the efficacy of the combined treatment of the present invention. The obtained data further demonstrate the general applicability of the combined treatments, in terms of chemosensitizers, chemotherapeutic drugs, and tumor origins.

Mechanism:

In order to provide additional research support to the in vitro results shown above, as well as to the in vivo results presented hereinafter, the mechanism by which Lithium enhances the cytotoxicity of both free and carrier-entrapped drug was explored. As in the cytotoxicity case, verapamil was used as a benchmark.

The Lithium mechanism for enhancing the cytotoxicity of the chemotherapeutic drug was evaluated by measuring its influence on the drug efflux, using the experimental procedures described hereinabove.

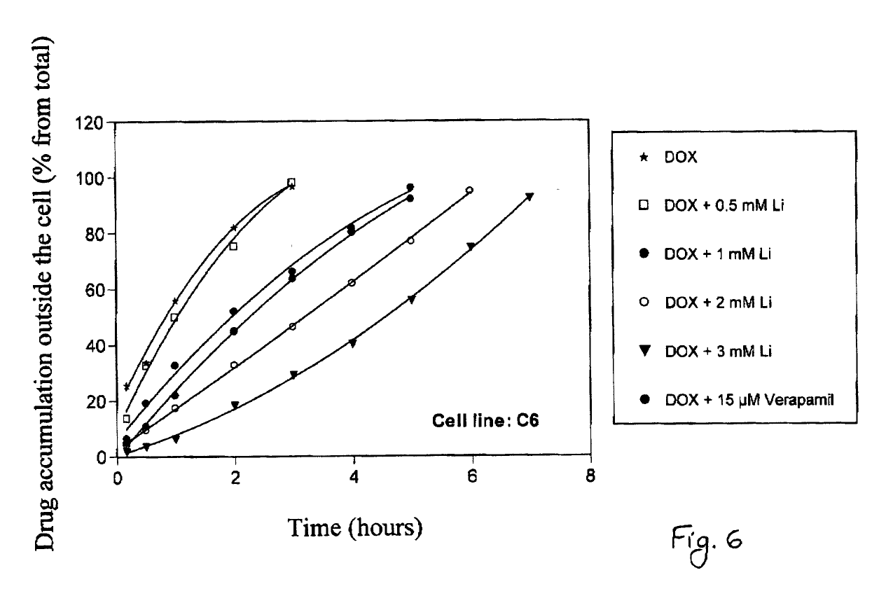

FIG. 6 presents the results obtained for the drug efflux following treatments with doxorubicin and various doses of Lithium or a selected verapamil dose, in cultures of C6 cells. The results are reported as the percentages of the drug that diffused into the external media (from the total drug quantity in the system at time=0 of the experiment, namely, the intracellular load), at different time points.

As is shown in FIG. 6, in the absence of a chemosensitizer, the cell is depleted from all the drug within 3 hours, in what appears to be a biphasic efflux. Moreover, it is shown in FIG. 6 that about 30% of the total intracellular drug load has already diffused out of the cell at the first time point measured, namely, after 5 minutes.

In the presence of verapamil, at the typical dose of 15 μM, the drug efflux was considerably slower, and 5 hours were needed for complete depletion of the cell. Furthermore, a significant reduction of the amount of the drug that diffused out at a 5 minutes time period (10%), as compared with an absence of a chemosensitizer, was observed.

Similar effects, namely increased time of complete depletion and decreased magnitude of the fast-phase efflux, were observed with Lithium. However, as a span of Lithium concentrations was tested, the obtained results indicate that these effects are directly dependent on the Lithium concentration. FIG. 6 clearly shows that the fraction of the fast-phase efflux was substantially decreased as the Lithium concentration was increased and was completely abolished with a 3 mM Lithium dose. The time of complete depletion was increased at increased Lithium concentration, reaching 7 hours at 3 mM Lithium.

The obtained results further show that the data obtained with 15 μM verapamil coincides with the data obtained with 1 mM Lithium. This feature fits the findings obtained in the cytotoxicity studies, shown in FIG. 3, as described hereinabove.

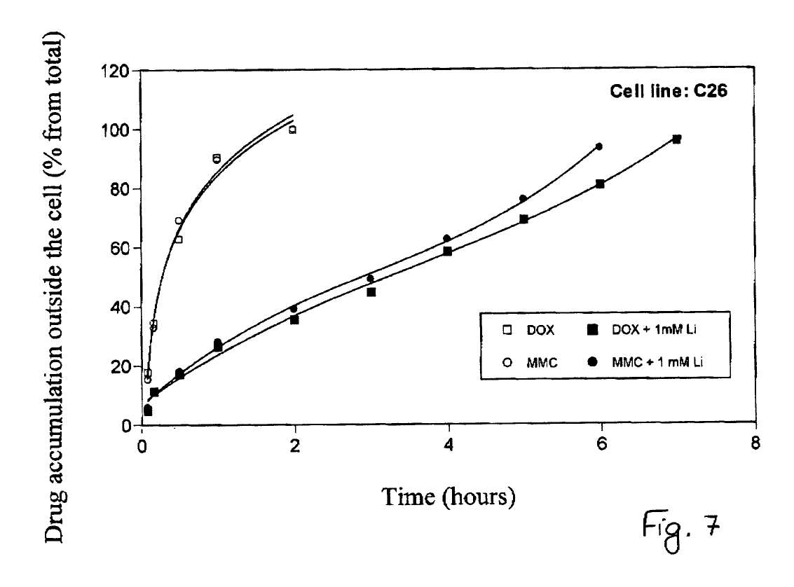

FIG. 7 presents the results obtained for the drug efflux, following treatments of with doxorubicin or MMC, in the presence of 1 mM Lithium, in cultures of C26 cells. Similar effects of increased time of complete depletion and decreased fraction of the fast-phase efflux, in the presence of Lithium, were observed.

The drug efflux data presented in FIGS. 6 and 7 provides substantial experimental support for the activity of Lithium as a chemosensitizer. The data further demonstrate that the chemosensitization activity of Lithium is not restricted to a single chemotherapeutic drug, nor to a single cell line. As is mentioned hereinabove, these results provide additional support to the in vitro cytotoxicity data and the in vivo data presented hereinafter.

In Vivo Studies:

35 mice, inoculated with tumor cells, were divides into groups and each group received a different treatment, as described hereinabove and is presented in Table 2 above. The animal survival was monitored continuously, from the first day of tumor inoculation.

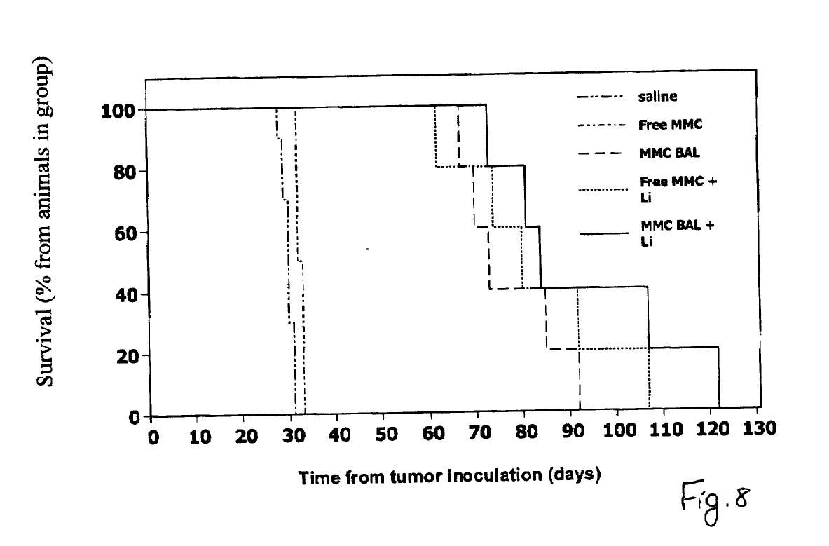

FIG. 8 presents the animal survival, expressed in percentages of the total number of animals in the group.

As shown in FIG. 8, the animals that received saline (group 1) or free drug (group 2) started dying on day 29 and were all dead on day 33. These results primarily indicate that the C26 tumor cells are drug resistant cells also in vivo, confirming that these cells provided a suitable in vivo model for testing the MDR reversal activity of the combined treatment of the present invention.

The animals that received a known treatment of a free drug and Lithium as a chemosensitizer (group 3), started dying at day 62, while 20% were still alive at day 92. The median was day 82.

The animals that received the drug entrapped in a targeted carrier, without a chemosensitizer (group 4), started dying at day 66 and were all dead at day 92. The median was day 74.

The animals that received the combined treatment, namely the drug entrapped in a targeted carrier and Lithium (group 5), were the best survivors. These animals sty dying on day 74, and 40% were still alive at day 92, demonstrating the synergy achieved by the treatment of the present invention.

The in vitro cytotoxicity data, the in vitro mechanistic data, and the in vivo data demonstrate the efficacy of the combined treatment of the present invention. Hence, these data demonstrate that the combined therapy presented herein generates a synergistic effect in treating MDR in vivo and hence provide a significant improvement of the clinical outcome of the chemotherapeutic treatment.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A pharmaceutical composition comprising:
a chemotherapeutically effective amount of at least one chemotherapeutic agent, said at least one chemotherapeutic agent being entrapped in a drug carrier; and
a chemosensitizing effective amount of at least one chemosensitizing agent.

2. The pharmaceutical composition of claim 1, wherein said drug carrier is a targeted drug carrier having an affinity to cancer cells.

3. The pharmaceutical composition of claim 2, wherein said drug carrier comprises liposomes.

4. The pharmaceutical composition of claim 3, wherein said liposomes are bioadhesive liposomes.

5. The pharmaceutical composition of claim 4, wherein said bioadhesive liposomes comprise a hyaluronic acid.

6. The pharmaceutical composition of claim 1, wherein said at least one chemotherapeutic agent is selected from the group consisting of an alkylating agent, an antimetabolite, a natural product, a miscellaneous agent, a hormone and an antagonist.

7. The pharmaceutical composition of claim 1, wherein said at least one chemosensitizing agent is selected from the group consisting of a calcium channel blocker, a calmodulin inhibitor, an indole alkaloid, a quinolines, a lysosomotropic agent, a steroid, a triparanol analog, a detergent, a cyclic peptide antibiotic, a psychotherapeutic agent, a cyclic psychotropic agent, and a 3-aryloxy-3-phenylpropylamine.

8. The pharmaceutical composition of claim 7, wherein said at least one chemosensitizing agent comprises a calcium channel blocker and said calcium channel blocker is verapamil.

9. The pharmaceutical composition of claim 1, wherein said chemosensitizing agent is Lithium.

10. The pharmaceutical composition of claim 1, wherein said chemotherapeutic agent is doxorubicin.

11. The pharmaceutical composition of claim 1, wherein said chemotherapeutic agent is mitomycin C.

12. The pharmaceutical composition of claim 3, wherein said chemotherapeutic agent is selected from the group consisting of doxorubicin and mitomycin C.

13. The pharmaceutical composition of claim 4, wherein said chemotherapeutic agent is selected from the group consisting of doxorubicin and mitomycin C.

14. The pharmaceutical composition of claim 12, wherein said chemosensitizing agent is selected from the group consisting of Lithium and verapamil.

15. The pharmaceutical composition of claim 13, wherein said chemosensitizing agent is selected from the group consisting of Lithium and verapamil.

16. The pharmaceutical composition of claim 1, further comprising a chemoprotective amount of at least one chemoprotecting agent.

17. The pharmaceutical composition of claim 16, wherein said at least one chemoprotecting agent is selected from the group consisting of a bis-dioxopiperazine, a D-methionine, a thiol-containing compound, a selenol-containing compounds, an amifostine, an ergotamine, a pyrridoxine, a lymphotoxin, a DPPE analog and a psychotropic agent.

18. The pharmaceutical composition of claim 1, identified for use in cancer therapy.

19. The pharmaceutical composition of claim 1, packaged in a container and identified in print on or in said container for use in cancer therapy.

20. A pharmaceutical composition comprising:
a chemotherapeutically effective amount of at least one chemotherapeutic agent, said at least one chemotherapeutic agent being entrapped in a drug carrier;
a chemosensitizing effective amount of at least one chemosensitizing agent; and
a chemoprotective effective amount of at least one chemoprotecting agent.

21. The pharmaceutical composition of claim 20, wherein said drug carrier is a targeted drug carrier having an affinity to cancer cells.

22. The pharmaceutical composition of claim 21, wherein said drug carrier comprises liposomes.

23. The pharmaceutical composition of claim 22, wherein said liposomes are bioadhesive liposomes.

24. The pharmaceutical composition of claim 23, wherein said bioadhesive liposomes comprise a hyaluronic acid.

25. The pharmaceutical composition of claim 20, wherein said at least one chemotherapeutic agent is selected from the group consisting of an alkylating agent, an antimetabolite, a natural product, a miscellaneous agent, a hormone and an antagonist.

26. The pharmaceutical composition of claim 20, wherein said at least one chemosensitizing agent is selected from the group consisting of a calcium channel blocker, a calmodulin inhibitor, an indole alkaloid, a quinolines, a lysosomotropic agent, a steroid, a triparanol analog, a detergent, a cyclic peptide antibiotic, a psychotherapeutic agent, a cyclic psychotropic agent and a 3-aryloxy-3-phenylpropylamine.

27. The pharmaceutical composition of claim 26, wherein said at least one chemosensitizing agent comprises a calcium channel blocker and said calcium channel blocker is verapamil.

28. The pharmaceutical composition of claim 20, wherein said chemosensitizing agent is Lithium.

29. The pharmaceutical composition of claim 20, wherein said chemotherapeutic agent is doxorubicin.

30. The pharmaceutical composition of claim 20, wherein said chemotherapeutic agent is mitomycin C.

31. The pharmaceutical composition of claim 22, wherein said chemotherapeutic agent is selected from the group consisting of doxorubicin and mitomycin C.

32. The pharmaceutical composition of claim 23, wherein said chemotherapeutic agent is selected from the group consisting of doxorubicin and mitomycin C.

33. The pharmaceutical composition of claim 31, wherein said chemosensitizing agent is selected from the group consisting of Lithium and verapamil.

34. The pharmaceutical composition of claim 32, wherein said chemosensitizing agent is selected from the group consisting of Lithium and verapamil.

35. The pharmaceutical composition of claim 20, wherein said at least one chemoprotecting agent is selected from the group consisting of a bis-dioxopiperazine, a D-methionine, a thiol-containing compound, a selenol-containing compounds an amifostine, an ergotamine, a pyrridoxine, a lymphotoxin, a DPPE analog and a psychotropic agent.

36. The pharmaceutical composition of claim 20, identified for use in cancer therapy.

37. The pharmaceutical composition of claim 20, packaged in a container and identified in print on or in said container for use in cancer therapy.

38. A pharmaceutical composition comprising:
a chemotherapeutically effective amount of at least one chemotherapeutic agent, said at least one chemotherapeutic agent being entrapped in a drug carrier; and
a chemoprotective amount of at least one chemoprotecting agent.

39. The pharmaceutical composition of claim 38, wherein said drug carrier is a targeted drug carrier having an affinity to cancer cells.

40. The pharmaceutical composition of claim 39, wherein said drug carrier comprises liposomes.

41. The pharmaceutical composition of claim 40, wherein said liposomes are bioadhesive liposomes.

42. The pharmaceutical composition of claim 41, wherein said bioadhesive liposomes comprise a hyaluronic acid.

43. The pharmaceutical composition of claim 38, wherein said at least one chemotherapeutic agent is selected from the group consisting of an alkylating agent, an antimetabolite, a natural product, a miscellaneous agent, a hormone and an antagonist.

44. The pharmaceutical composition of claim 38, wherein said chemotherapeutic agent is doxorubicin.

45. The pharmaceutical composition of claim 38, wherein said chemotherapeutic agent is mitomycin C.

46. The pharmaceutical composition of claim 40, wherein said chemotherapeutic agent is selected from the group consisting of doxorubicin and mitomycin C.

47. The pharmaceutical composition of claim 41, wherein said chemotherapeutic agent is selected from the group consisting of doxorubicin and mitomycin C.

48. The pharmaceutical composition of claim 38, wherein said at least one chemoprotecting agent is selected from the group consisting of a bis-dioxopiperazine, a D-methionine, a thiol-containing compound, a selenol-containing compounds an amifostine, an ergotamine, a pyrridoxine, a lymphotoxin, a DPPE analog and a psychotropic agent.

49. The pharmaceutical composition of claim 38, further comprising a chemoprotective amount of at least one chemosensitizing agent.

50. The pharmaceutical composition of claim 49, wherein said at least one chemosensitizing agent is selected from the group consisting of a calcium channel blocker, a calmodulin inhibitor, an indole alkaloid, a quinolines, a lysosomotropic agent, a steroid, a triparanol analog, a detergent, a cyclic peptide antibiotic, a psychotherapeutic agent, a cyclic psychotropic agent and a 3-aryloxy-3-phenylpropylamine.

51. The pharmaceutical composition of claim 50, wherein said at least one chemosensitizing agent comprises a calcium channel blocker and said calcium channel blocker is verapamil.

52. The pharmaceutical composition of claim 49, wherein said chemosensitizing agent is Lithium.

53. The pharmaceutical composition of claim 38, identified for use in cancer therapy.

54. The pharmaceutical composition of claim 38, packaged in a container and identified in print on or in said container for use in cancer therapy.

55. A pharmaceutical kit comprising, as a chemotherapeutically active ingredient, at least one chemotherapeutic agent being entrapped in a drug carrier, and at least one chemosensitizing agent, wherein said at least one chemotherapeutic agent and said at least one chemosensitizing agent are individually packaged within the pharmaceutical kit.

56. The pharmaceutical kit of claim 55, wherein said drug carrier is a targeted drug carrier having an affinity to cancer cells.

57. The pharmaceutical kit of claim 56, wherein said drug carrier comprises liposomes.

58. The pharmaceutical kit of claim 57, wherein said liposomes are bioadhesive liposomes.

59. The pharmaceutical kit of claim 58, wherein said bioadhesive liposomes comprise a hyaluronic acid.

60. The pharmaceutical kit of claim 55, wherein said at least one chemotherapeutic agent is selected from the group consisting of an alkylating agent, an antimetabolite, a natural product, a miscellaneous agent a hormone and an antagonist.

61. The pharmaceutical kit of claim 55, wherein said at least one chemosensitizing agent is selected from the group consisting of a calcium channel blocker, a calmodulin inhibitor, an indole alkaloid, a quinolines, a lysosomotropic agent, a steroid, a triparanol analog, a detergent, a cyclic peptide antibiotic, a psychotherapeutic agent, a cyclic psychotropic agent and a 3-aryloxy-3-phenylpropylamine.

62. The pharmaceutical kit of claim 61, wherein said at least one chemosensitizing agent comprises a calcium channel blocker and said calcium channel blocker is verapamil.

63. The pharmaceutical kit of claim 56, wherein said chemosensitizing agent is Lithium.

64. The pharmaceutical kit of claim 55, wherein said chemotherapeutic agent is doxorubicin.

65. The pharmaceutical kit of claim 55, wherein said chemotherapeutic agent is mitomycin C.

66. The pharmaceutical kit of claim 57, wherein said chemotherapeutic agent is selected from the group consisting of doxorubicin and mitomycin C.

67. The pharmaceutical kit of claim 58, wherein said chemotherapeutic agent is selected from the group consisting of doxorubicin and mitomycin C.

68. The pharmaceutical kit of claim 66, wherein said chemosensitizing agent is selected from the group consisting of Lithium and verapamil.

69. The pharmaceutical composition of claim 67, wherein said chemosensitizing agent is selected from the group consisting of Lithium and verapamil.

70. The pharmaceutical kit of claim 55, identified in print for use in cancer therapy.

71. A pharmaceutical kit comprising, as a chemotherapeutically active ingredient, at least one chemotherapeutic agent being entrapped in a drug carrier, and at least one chemoprotecting agent, wherein said at least one chemotherapeutic agent and said at least one chemoprotecting agent are individually packaged within the pharmaceutical kit.

72. The pharmaceutical kit of claim 71, wherein said drug carrier is a targeted drug carrier having an affinity to cancer cells.

73. The pharmaceutical kit of claim 72, wherein said drug carrier comprises liposomes.

74. The pharmaceutical kit of claim 73, wherein said liposomes are bioadhesive liposomes.

75. The pharmaceutical kit of claim 74, wherein said bioadhesive liposomes comprise a hyaluronic acid.

76. The pharmaceutical kit of claim 71, wherein said at least one chemotherapeutic agent is selected from the group consisting of an alkylating agent, an antimetabolite, a natural product, a miscellaneous agent, a hormone and an antagonist.

77. The pharmaceutical kit of claim 71, wherein said chemotherapeutic agent is doxorubicin.

78. The pharmaceutical kit of claim 71, wherein said chemotherapeutic agent is mitomycin C.

79. The pharmaceutical kit of claim 71, wherein said chemotherapeutic agent is selected from the group consisting of doxorubicin and mitomycin C.

80. The pharmaceutical kit of claim 74, wherein said chemotherapeutic agent is selected from the group consisting of doxorubicin and mitomycin C.

81. The pharmaceutical kit of claim 71, wherein said at least one chemoprotecting agent is selected from the group consisting of a bis-dioxopiperazine, a D-methionine, a thiol-containing compound, a selenol-containing compounds an amifostine, an ergotamine, a pyrridoxine, a lymphotoxin, a DPPE analog and a psychotropic agent.

82. The pharmaceutical kit of claim 80, identified in print for use in cancer therapy.

83. A pharmaceutical kit comprising, as a chemotherapeutically active ingredient, at least one chemotherapeutic agent being entrapped in a drug carrier, at least one chemosensitizing agent and at least one chemoprotecting agent, wherein said at least one chemotherapeutic agent, said at least one chemosensitizing agent and said at least one chemoprotecting agent are individually packaged within the pharmaceutical kit.

84. The pharmaceutical kit of claim 83, wherein said drug carrier is a targeted drug carrier having an affinity to cancer cells.

85. The pharmaceutical kit of claim 84, wherein said drug carrier comprises liposomes.

86. The pharmaceutical kit of claim 85, wherein said liposomes are bioadhesive liposomes.

87. The pharmaceutical kit of claim 86, wherein said bioadhesive liposomes comprise a hyaluronic acid.

88. The pharmaceutical kit of claim 83, wherein said at least one chemotherapeutic agent is selected from the group consisting of an alkylating agent, an antimetabolite, a natural product, a miscellaneous agent, a hormone and an antagonist.

89. The pharmaceutical kit of claim 83, wherein said at least one chemosensitizing agent is selected from the group consisting of a calcium channel blocker, a calmodulin inhibitor, an indole alkaloid, a quinolines, a lysosomotropic agent, a steroid, a triparanol analog, a detergent, a cyclic peptide antibiotic, a psychotherapeutic agent, a cyclic psychotropic agent and a 3-aryloxy-3-phenylpropylamine.

90. The pharmaceutical kit of claim 89, wherein said at least one chemosensitizing agent comprises a calcium channel blocker and said calcium channel blocker is verapamil.

91. The pharmaceutical kit of claim 83, wherein said chemosensitizing agent is Lithium.

92. The pharmaceutical kit of claim 83, wherein said chemotherapeutic agent is doxorubicin.

93. The pharmaceutical kit of claim 83, wherein said chemotherapeutic agent is mitomycin C.

94. The pharmaceutical kit of claim 85, wherein said chemotherapeutic agent is selected from the group consisting of doxorubicin and mitomycin C.

95. The pharmaceutical kit of claim 86, wherein said chemotherapeutic agent is selected from the group consisting of doxorubicin and mitomycin C.

96. The pharmaceutical kit of claim 94, wherein said chemosensitizing agent is selected from the group consisting of Lithium and verapamil.

97. The pharmaceutical composition of claim 95, wherein said chemosensitizing agent is selected from the group consisting of Lithium and verapamil.

98. The pharmaceutical kit of claim 83, wherein said at least one chemoprotecting agent is selected from the group consisting of a bis-dioxopiperazine, a D-methionine, a thiol-containing compound, a selenol-containing compounds an amifostine, an ergotamine, a pyrridoxine, a lymphotoxin, a DPPE analog and a psychotropic agent.

99. The pharmaceutical kit of claim 83, identified in print for use in cancer therapy.

100. A method of treating cancer in a subject in need thereof, the method comprising:
administering to said subject a chemotherapeutically effective amount of at least one chemotherapeutic agent being entrapped in a drug carrier; and
administering to said subject a chemosensitizing effective amount of at least one chemosensitizing agent.

101. The method of claim 100, wherein the administration of said chemotherapeutic agent is performed prior to, concomitant with or following the administration of said chemosensitizing agent.

102. The method of claim 100, further comprising:
administering to said subject a chemoprotective effective amount of at least one chemoprotective agent.

103. The method of claim 102, wherein the administration of said chemotherapeutic agent is performed prior to, concomitant with or following the administration of said chemosensitizing agent and/or the administration of said chemoprotective agent.

104. The method of claim 102, wherein the administration of said chemosensitizing agent is performed prior to, concomitant with or following the administration of said chemotherapeutic agent and/or the administration of said chemoprotective agent.

105. The method of claim 102, wherein the administration of said chemoprotecting agent is performed prior to, concomitant with or following the administration of said chemosensitizing agent and/or the administration of said chemotherapeutic agent.

106. The method of claim 100, wherein said drug carrier is a targeted drug carrier having an affinity to cancer cells.

107. The method of claim 106, wherein said drug carrier comprises liposomes.

108. The method of claim 107, wherein said liposomes are bioadhesive liposomes.

109. The method of claim 108, wherein said bioadhesive liposomes comprise a hyaluronic acid.

110. The method of claim 100, wherein said at least one chemotherapeutic agent is selected from the group consisting of an alkylating agent, an antimetabolite, a natural product, a miscellaneous agent, a hormone and an antagonist.

111. The method of claim 100, wherein said at least one chemosensitizing agent is selected from the group consisting of a calcium channel blocker, a calmodulin inhibitor, an indole alkaloid, a quinolines, a lysosomotropic agent, a steroid, a triparanol analog, a detergent, a cyclic peptide antibiotic, a psychotherapeutic agent, a cyclic psychotropic agent and a 3-aryloxy-3-phenylpropylamine.

112. The method of claim 111, wherein said at least one chemosensitizing agent comprises a calcium channel blocker and said calcium channel blocker is verapamil.

113. The method of claim 100, wherein said chemosensitizing agent is Lithium.

114. The method of claim 100, wherein said chemotherapeutic agent is doxorubicin.

115. The method of claim 100, wherein said chemotherapeutic agent is mitomycin C.

116. The method of claim 107, wherein said chemotherapeutic agent is selected from the group consisting of doxorubicin and mitomycin C.

117. The method of claim 108, wherein said chemotherapeutic agent is selected from the group consisting of doxorubicin and mitomycin C.

118. The method of claim 116, wherein said chemosensitizing agent is selected from the group consisting of Lithium and verapamil.

119. The method of claim 117, wherein said chemosensitizing agent is selected from the group consisting of Lithium and verapamil.

120. The method of claim 102, wherein said at least one chemoprotecting agent is selected from the group consisting of a bis-dioxopiperazine, a D-methionine, a thiol-containing compound, a selenol-containing compounds an amifostine, an ergotamine, a pyrridoxine, a lymphotoxin, a DPPE analog and a psychotropic agent.

121. A method of treating cancer cells in an subject, the method comprising:
administering to said subject a chemotherapeutically effective amount of at least one chemotherapeutic agent being entrapped in a drug carrier;
administering to said subject a chemosensitizing effective amount of at least one chemosensitizing agent; and
administering to said subject a chemoprotective effective amount of at least one chemoprotective agent.

122. The method of claim 121, wherein the administration of said chemotherapeutic agent is performed prior to, concomitant with or following the administration of said chemosensitizing agent and/or the administration of said chemoprotective agent.

123. The method of claim 121, wherein the administration of said chemosensitizing agent is performed prior to, concomitant with or following the administration of said chemotherapeutic agent and/or the administration of said chemoprotective agent.

124. The method of claim 121, wherein the administration of said chemoprotecting agent is performed prior to, concomitant with or following the administration of said chemosensitizing agent and/or the administration of said chemotherapeutic agent.

125. The method of claim 121, wherein said drug carrier is a targeted drug carrier having an affinity to cancer cells.

126. The method of claim 125, wherein said drug carrier comprises liposomes.

127. The method of claim 126, wherein said liposomes are bioadhesive liposomes.

128. The method of claim 127, wherein said bioadhesive liposomes comprise a hyaluronic acid.

129. The method of claim 121, wherein said at least one chemotherapeutic agent is selected from the group consisting of an alkylating agent, an antimetabolite, a natural product, a miscellaneous agent, a hormone and an antagonist.

130. The method of claim 121, wherein said at least one chemosensitizing agent is selected from the group consisting of a calcium channel blocker, a calmodulin inhibitor, an indole alkaloid, a quinolines, a lysosomotropic agent, a steroid, a triparanol analog, a detergent, a cyclic peptide antibiotic, a psychotherapeutic agent, a cyclic psychotropic agent and a 3-aryloxy-3-phenylpropylamine.

131. The method of claim 130, wherein said at least one chemosensitizing agent comprises a calcium channel blocker and said calcium channel blocker is verapamil.

132. The method of claim 121, wherein said chemosensitizing agent is Lithium.

133. The method of claim 121, wherein said chemotherapeutic agent is doxorubicin.

134. The method of claim 121, wherein said chemotherapeutic agent is mitomycin C.

135. The method of claim 126, wherein said chemotherapeutic agent is selected from the group consisting of doxorubicin and mitomycin C.

136. The method of claim 127, wherein said chemotherapeutic agent is selected from the group consisting of doxorubicin and mitomycin C.

137. The method of claim 135, wherein said chemosensitizing agent is selected from the group consisting of Lithium and verapamil.

138. The method of claim 136, wherein said chemosensitizing agent is selected from the group consisting of Lithium and verapamil.

139. The method of claim 121, wherein said at least one chemoprotecting agent is selected from the group consisting of a bis-dioxopiperazine, a D-methionine, a thiol-containing compound, a selenol-containing compounds an amifostine, an ergotamine, a pyrridoxine, a lymphotoxin, a DPPE analog and a psychotropic agent.

140. A method of treating cancer in a subject in need thereof, the method comprising:
administering to said subject a chemotherapeutically effective amount of at least one chemotherapeutic agent being entrapped in a drug carrier; and
administering to said subject a chemoprotective effective amount of at least one chemoprotecting agent.

141. The method of claim 140, wherein the administration of said chemotherapeutic agent is performed prior to, concomitant with or following the administration of said chemoprotecting agent.

142. The method of claim 140, further comprising:
administering to said subject a chemosensitizing effective amount of at least one chemosensitizing agent.

143. The method of claim 142, wherein the administration of said chemotherapeutic agent is performed prior to, concomitant with or following the administration of said chemosensitizing agent and/or the administration of said chemoprotective agent.

144. The method of claim 142, wherein the administration of said chemosensitizing agent is performed prior to, concomitant with or following the administration of said chemotherapeutic agent and/or the administration of said chemoprotective agent.

145. The method of claim 142, wherein the administration of said chemoprotecting agent is performed prior to, concomitant with or following the administration of said chemosensitizing agent and/or the administration of said chemotherapeutic agent.

146. The method of claim 140, wherein said drug carrier is a targeted drug carrier having an affinity to cancer cells.

147. The method of claim 146, wherein said drug carrier comprises liposomes.

148. The method of claim 147, wherein said liposomes are bioadhesive liposomes.

149. The method of claim 148, wherein said bioadhesive liposomes comprise a hyaluronic acid.

150. The method of claim 140, wherein said at least one chemotherapeutic agent is selected from the group consisting of an alkylating agent an antimetabolite, a natural product, a miscellaneous agent, a hormone and an antagonist.

151. The method of claim 140, wherein said chemotherapeutic agent is doxorubicin.

152. The method of claim 140, wherein said chemotherapeutic agent is mitomycin C.

153. The method of claim 147, wherein said chemotherapeutic agent is selected from the group consisting of doxorubicin and mitomycin C.

154. The method of claim 148, wherein said chemotherapeutic agent is selected from the group consisting of doxorubicin and mitomycin C.

155. The method of claim 140, wherein said at least one chemoprotecting agent is selected from the group consisting of a bis-dioxopiperazine, a D-methionine, a thiol-containing compound, a selenol-containing compounds an amifostine, an ergotamine, a pyrridoxine, a lymphotoxin, a DPPE analog and a psychotropic agent.

156. The method of claim 142, wherein said at least one chemosensitizing agent is selected from the group consisting of a calcium channel blocker, a calmodulin inhibitor, an indole alkaloid, a quinolines, a lysosomotropic agent, a steroid, a triparanol analog, a detergent, a cyclic peptide antibiotic, a psychotherapeutic agent, a cyclic psychotropic agent and a 3-aryloxy-3-phenylpropylamine.

157. The method of claim 156, wherein said at least one chemosensitizing agent comprises a calcium channel blocker and said calcium channel blocker is verapamil.

158. The method of claim 142, wherein said chemosensitizing agent is Lithium.

159. A method chemosensitizing cancer cells in a subject in need thereof, the method comprising administering to the subject a chemosensitizing effective amount of Lithium.

160. A method chemosensitizing MDR cancer cells in a subject in need thereof, the method comprising administering to the subject a chemosensitizing effective amount of Lithium.

161. A method of treating cancer in a subject in need thereof, the method comprising administering to the subject a chemosensitizing effective amount of Lithium and a chemotherapeutically effective amount of a chemotherapeutic agent.

162. A method of treating MDR cancer in a subject in need thereof, the method comprising administering to the subject a chemosensitizing effective amount of Lithium and a chemotherapeutically effective amount of a chemotherapeutic agent.

163. A pharmaceutical composition for treating cancer comprising, as a chemosensitizing agent a chemosensitizing effective amount of Lithium and a chemotherapeutically effective amount of a chemotherapeutic agent.

164. A pharmaceutical composition for treating MDR cancer comprising, as a chemosensitizing agent a chemosensitizing effective amount of Lithium and a chemotherapeutically effective amount of a chemotherapeutic agent.

165. A pharmaceutical composition for chemosensitizing cancer cells in a subject in need thereof, comprising, as a chemosensitizing agent, a chemosensitizing effective amount of Lithium, the pharmaceutical composition is packaged in a package and is identified in print in or on said package for use in chemosensitizing.

166. A pharmaceutical kit comprising, as a chemotherapeutically active ingredient, at least one chemotherapeutic agent, and Lithium, wherein said at least one chemotherapeutic agent and said Lithium are individually packaged within the pharmaceutical kit.
